(19) United States
(12) Patent Application Publication
(10) Pub. No.: US 2026/0108163 A1
(43) Pub. Date: Apr. 23, 2026

(54) METHOD AND APPARATUS FOR ADAPTIVELY DETECTING AN ACUTE HYPOTENSION EPISODE EVENT OF A PATIENT

(71) Applicant: NEC Corporation, Minato-ku, Tokyo (JP)

(72) Inventors: Ni Ni SOE, Singapore (SG); Charles Chi Hin CHOY, Singapore (SG)

(73) Assignee: NEC Corporation, Minato-ku, Tokyo (JP)

(21) Appl. No.: 18/294,570

(22) PCT Filed: Aug. 5, 2022
(86) PCT No.: PCT/JP2022/030116

§ 371 Date: Feb. 2, 2024

(30) Foreign Application Priority Data
Aug. 10, 2021 (SG) 10202108729V

(51) Int. Cl. A61B 5/022; A61B 5/00; A61B 5/0205

(52) U.S. Cl. CPC A61B 5/022; A61B 5/0205; A61B 5/7246; A61B 5/7275

(57) ABSTRACT

The present disclosure provides a method and an apparatus for adaptively detecting an acute hypotension episode (AHE) event of a patient, the method comprising: processing, by a processor, a signal relating to the patient to determine at least one mean arterial pressure (MAP) threshold value for the patient (101); and detecting, by the processor, an AHE event of the patient based on the at least one MAP threshold value (102).

### TECHNICAL FIELD

The present invention relates broadly, but not exclusively, to a method and an apparatus for adaptive detecting an acute hypotension episode (AHE) event of a patient.

### BACKGROUND ART

Hypotension is a sign of cardio circulatory dysfunction. Profound hypotension is common in patients having surgery and in critically ill patients. In patients who are undergoing major surgical procedure, intraoperative hypotension commonly accompanies with general anesthesia. It is also associated with postoperative adverse complications.

The duration and severity of acute hypotension episode (AHE) event is a critical event to a patient who is associated with organ hypoperfusion and dysfunction, and can lead to irreversible shock. It represents the beginning of a possible pathway to exitus.

An AHE event requires effective and prompt intervention. In other words, AHE event has to be treated reactively after low blood pressure values have already occurred. When detected in time, an appropriate intervention can significantly lower the risks on a patient.

### SUMMARY OF INVENTION

### Technical Problem

To date, monitoring systems in the operating room or the intensive care unit enable numeral and waveform blood pressure data to be processed and displayed continuously in real time. However, management of AHE event mainly consists of reactive treatment with pressors and fluids after low blood pressure values have already occurred. In particular, clinicians react to an absolute blood pressure (e.g. mean arterial pressure (MAP)) threshold value or short term blood pressure changes to detect an AHE event of a patient, and reactively treat the patient to avoid profound or sustained hypotension event. However, low blood pressure values typically occur late in the process of hemodynamic instability, that is, when global cardiovascular dynamics have already been markedly altered and compensatory mechanisms are exhausted.

Further, prediction of an AHE event before it becomes clinically apparent would allow clinician to treat the event pre-emptively, thereby reducing the duration and severity of the hypotension. AHE event can now be predicted in scale of minutes before it occurs based on blood pressure waveform using machine-learning algorithms and a hard threshold value of blood pressure (e.g. MAP). However, analyzing the complex cardiovascular system of different patients using a single, hard blood pressure threshold value is a challenge because cardiovascular physiology is highly interdependent, works within complicated networks and is influenced by compensatory mechanisms within each individual patient.

Therefore, there is a need that provide a personalized MAP threshold value for each patient for early identification of AHE events, or a prediction of an AHE event of the patient before it becomes clinically apparent. This would allow clinician to apply pre-emptive treatment strategies and reduce occurrence, duration, and severity of AHE events of the patient.

Herein disclosed are embodiments of a device and methods for adaptively detecting an AHE event of a patient that addresses one or more of the above problems.

Furthermore, other desirable features and characteristics will become apparent from the subsequent detailed description and the appended claims, taken in conjunction with the accompanying drawings and this background of the disclosure.

### Solution to Problem

In a first aspect, the present disclosure provides a method for adaptively detecting an Acute Hypotension Episode (AHE) event of a patient, comprising: processing, by a processor, a signal relating to the patient to determine at least one mean arterial pressure (MAP) threshold value for the patient; and detecting, by the processor, an AHE event of the patient based on the at least one MAP threshold value.

In a second aspect, the present disclosure provides an apparatus for adaptively detecting an AHE event of a patient, comprising: at least one processor; and at least one memory including computer program code; the at least one memory and the computer program code configured to, with at least one processor, cause the apparatus at least to perform the method according to the first aspect.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### Advantageous Effects of Invention

According to the present disclosure, it is possible to provide a device and methods for adaptively detecting an AHE event of a patient that addresses one or more of the above problems.

### BRIEF DESCRIPTION OF DRAWINGS

The accompanying Figures, where like reference numerals refer to identical or functionally similar elements throughout the separate views and which together with the detailed description below are incorporated in and form part of the specification, serve to illustrate various embodiments and to explain various principles and advantages in accordance with a present embodiment, by way of non-limiting example only.

Embodiments of the invention will be better understood and readily apparent to one of ordinary skill in the art from the following written description, by way of example only, and in conjunction with the drawings, in which:

FIG. 1 shows a flow chart illustrating a method for adaptively detecting an acute hypotension episode (AHE) event of a patient according to various embodiments of the present disclosure.

FIG. 2 shows a block diagram illustrating an apparatus for adaptively detecting an AHE event of a patient according to various embodiments of the present disclosure.

FIG. 3 shows a block diagram 300 illustrating a signal source 301 and a personalized AHE definition module 304 for determining a mean arterial pressure (MAP) threshold value for a patient according to an embodiment of the present disclosure.

FIG. 4 shows a flow chart illustrating a part of a method for determining a MAP threshold value for a patient according to an embodiment of the present disclosure.

FIG. 5 shows a mean θ against time t graph illustrating two different states s of a two-state model of a two-state model used for determining a MAP threshold value for a patient according to an embodiment of the present disclosure.

FIG. 6A shows a MAP waveform and a processed MAP waveform respectively according to an embodiment of the present disclosure.

FIG. 6B shows a MAP waveform and a processed MAP waveform respectively according to an embodiment of the present disclosure.

FIG. 7 shows a processed MAP waveform and personalized AHE parameters according to an embodiment of the present disclosure.

FIG. 8 shows a flow chart illustrating another part of a method for determining a MAP threshold value for a patient according to an embodiment of the present disclosure.

FIG. 9A shows various MAP waveforms and AHE events detected therein according to various embodiments of the present disclosure.

FIG. 9B shows various MAP waveforms and AHE events detected therein according to various embodiments of the present disclosure.

FIG. 10A shows various MAP waveforms and AHE events detected therein according to various embodiments of the present disclosure.

FIG. 10B shows various MAP waveforms and AHE events detected therein according to various embodiments of the present disclosure.

FIG. 10C shows various MAP waveforms and AHE events detected therein according to various embodiments of the present disclosure.

FIG. 11A shows various MAP waveforms and AHE events detected therein according to various embodiments of the present disclosure.

FIG. 11B shows various MAP waveforms and AHE events detected therein according to various embodiments of the present disclosure.

FIG. 11C shows various MAP waveforms and AHE events detected therein according to various embodiments of the present disclosure.

FIG. 12A shows various MAP waveforms and AHE events detected therein according to various embodiments of the present disclosure.

FIG. 12B shows various MAP waveforms and AHE events detected therein according to various embodiments of the present disclosure.

FIG. 12C shows various MAP waveforms and AHE events detected therein according to various embodiments of the present disclosure.

FIG. 13A shows various MAP waveforms and AHE events detected therein according to various embodiments of the present disclosure.

FIG. 13B shows various MAP waveforms and AHE events detected therein according to various embodiments of the present disclosure.

FIG. 13C shows various MAP waveforms and AHE events detected therein according to various embodiments of the present disclosure.

FIG. 13D shows various MAP waveforms and AHE events detected therein according to various embodiments of the present disclosure.

FIG. 14 shows a block diagram a hypotension prediction module for adaptively detecting and predicting an AHE event of a patient according to an embodiment of the present disclosure.

FIG. 15 shows a flow chart illustrating a part of a method of pre-processing a signal and extracting features of the signal for determining an prediction of an AHE event of a patient according to an embodiment of the present disclosure.

FIG. 16 shows a block diagram 1600 illustrating inputs and outputs of a stacking ensemble model based predictive modelling engine 1601 used for adaptive detecting and predicting an AHE event of a patient according to an embodiment of the present disclosure.

FIG. 17A shows various input data used for adaptively detecting and predicting an AHE event of a patient according to various embodiments of the present disclosure.

FIG. 17B shows a MAP waveform used as an input for adaptively detecting and predicting an AHE event of a patient according to an embodiment of the present disclosure.

FIG. 18 shows features extracted from signals relating to a plurality of patients used for model training according an embodiment of the present disclosure.

FIG. 19 shows a diagram illustrating a process of extracting at least one feature in a signal used for adaptively detecting and predicting an AHE event of a patient according an embodiment of the present disclosure.

FIG. 20 shows a block diagram illustrating an apparatus for detecting and predicting an AHE event of a patient according to an embodiment of the present disclosure.

FIG. 21 shows a flow diagram illustrating a process for detecting and predicting an AHE event of a patient according an embodiment of the present disclosure.

FIG. 22 shows a schematic diagram of an exemplary computing device suitable for use to execute the method in FIG. 1 and implement the apparatus in FIG. 2.

### DESCRIPTION OF EMBODIMENTS

### Terms Description

Patient—a patient may be a user or a person who is at home, being transmitted, for example via an ambulance or a medically equipped vehicle, to or currently admitted in the hospital, for example in accident and emergency department, that requires his/her mean arterial pressure (MAP) threshold value to be determined, an AHE event to be adaptively detected based on his/her MAP threshold value as well as a subsequent AHE event to be predicted.

Signal—a signal may be one or a combination of hemodynamic data such as systolic blood pressure (BPSys or SBP), heart rate (HR), respiratory rate (RESP), MAP, diastolic blood pressure (BPDia or DBP), pulse pressure (PP), blood oxygen saturation (SPO₂) and other clinical data that are, for example stored or received from an equipment or a measurement conducted in intensive care unit (ICU), emergency department (ED), input put by occupational therapy (OT), patient monitoring unit (PMU), and/or patient data from the perioperative departments, data from the ambulance or medically equipped vehicle, ward, and/or electronic medical records (EMR). Such data can be invasively acquired or non-invasively acquired from a patient directly or indirectly. In various embodiments below, MAP data or MAP value is used as a signal for detections and predictions of an AHE of a patient. It is appreciated that the method and the apparatus can be configured to use other hemodynamic or clinical data to achieve similar results.

Mean arterial pressure (MAP) threshold value—a MAP threshold value is used to compare against values of MAP waveform relating to a patient to detect an AHE event of the patient. Conventionally, a fixed MAP threshold of 60 or 65 mmHg is defined, and if the MAP waveform of any patient has MAP values lower than that fixed MAP threshold in 90% of the time within a 30-minute time period, an AHE event is detected. In contrast, various embodiments below provide a personalized AHE definition for different patients based on personalized MAP threshold values. In other words, different MAP threshold values defined for different patients for detections of respective personalized AHE events. The MAP threshold values are calculated using respective baselines and fluctuations of their own MAP waveforms. Such MAP threshold value(s) is a MAP value offset from MAP waveform baseline, where the offset amount depends on the fluctuations of patient's MAP waveform. Subjected to other determining factor, a personalized AHE is detected when the patient's MAP waveform crosses the threshold value(s), i.e. the patient's MAP has switched from one state to another, for example from out-of-control (OOC) state to an in-control or baseline state or the other way round.

Baseline—a baseline of a signal may refer to a value, a derived value (e.g an average value or a mean value of several values), point or portion of the signal that is used to compare against the remaining values, measurements or portions of the signal to determine a deviation of the signal and MAP threshold value(s) for a patient. In one example, a baseline is taken directly or derived from values or measurements in the first portion of the signal, e.g. within the first few seconds, minutes and hours of the signal. In another example, a baseline of signal can be set at a value, point or portion of the signal either automatically upon detecting similar measurements consecutively in such value, point or portion or manually by an operator or analyst. In various processing and calculations below, the baseline may be referred to a baseline measurement or θ₀.

Fluctuation—a fluctuation of a signal refers to a variance of values or measurements of the signal. In various processing and calculations below, the fluctuation is referred to var(zₜ).

Feature—a feature relates to an attribute of a signal, typically a sample or segmented portion of the signal. Such feature can be computed and represented by feature data and compared against other signal to determine if they exhibit similar feature, attribute or feature data. Examples of features include maximum power, mean power, min power, maximum frequency, mean frequency, median frequency, minimum frequency, maximum of segmented data, mean of segmented data, minimum of segmented data, variance of segmented data, skewness of segmented data, kurtosis of segmented data, RMS value of segmented data and etc. In one example, a prediction of an occurrence of an AHE event of a patient can be determined by comparing features data relating the patient against those of other patients with/without AHE.

Embodiments of the present invention will be described, by way of example only, with reference to the drawings. Like reference numerals and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “receiving”, “calculating”, “determining”, “updating”, “generating”, “initializing”, “outputting”, “receiving”, “retrieving”, “identifying”, “dispersing”, “authenticating” or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM mobile telephone system. The computer program when loaded and executed on such a computer effectively results in an apparatus that implements the steps of the preferred method.

### EXEMPLARY EMBODIMENTS

Various embodiments of the present disclosure relate to a method and an apparatus for adaptively detecting an AHE event of a patient.

Conventionally, a prediction of an AHE event is based on the definition of AHE on MAP value at or below 60 mmHg in 90% of the time within a 30 minutes time frame. This fixed hard threshold of 60 mmHg is applied to every patient. In addition, prediction models are based on binary class model such as binary classification or logistic regression and supervised learning using neural network.

Recently, the algorithm has been developed as a feature of the EV1000 and Hemosphere clinical monitoring platforms (Edwards Lifesciences). In particular, hypotension prediction index (GPI) value, ranging from 0 to 100, is calculated every 20 seconds and displayed on a monitor. The HPI value represents a probability of a patient developing a hypotensive event which defined to be a MAP value less than 65 mmHg for at least 1 minute. Once the HPI value exceeds 85, an audible alarm will be set off.

There are two main problems that have been identified on the conventional and existing AHE detection solutions: 1) use of conventional definition of hypotension based on a single MAP threshold value, e.g. 60 mmHg or 65 mmHg, which does not account for individual patients: 2) use of binary classification or logistic regression in the prediction system to provide a probability and only a yes/no information of the occurrence of AHE in pre-defined onset time.

In particular, regarding the first problem statement, some people live well with a systolic of 90 mmHg and others are hypotensive with a systolic of 100 mmHg or lower: whereas elderly with atherosclerosis and hypertension can be hypotensive at a systolic of 120 mmHg for example. For patients with a history of hypertension, a decrease in the MAP of more than 40 mmHg is considered hypotension, even though the pressure is still above 60 mmHg. However, hypertensive patients or elderly do require higher MAP level.

Further, MAP is calculated based on equation (1) below:

$$MAP = \frac{1 \times DBP + 1 \times SBP}{3} \qquad equation ( 1 )$$

where DBP is diastolic blood pressure, SBP is systolic blood pressure. It is noted that MAP is an indicator of organ perfusion. There is no minimal MAP that ensures an adequate perfusion of all organs since the critical value of MAP is different for each organ.

Autoregulation threshold values vary among organs as well as between individuals. To increase MAP and obtain the target MAP, fluids (in shock states with decreased venous return) and pressors are required. A high target MAP value requires a higher load of pressor drug and may induce excessive arterial systemic vasoconstriction, which in may induce organ ischemia. Importantly, individual patients have specific baseline of MAP and they should not be considered under a same group. This is further supported by the European Society of Intensive Care Medicine who provided a new statement on their up to date consensus on individualizing blood pressure targets in treating shock.

Regarding the second problem statement, the existing prediction model only provides a yes/no information of the occurrence of an AHE event of a patient and does not mention a severity level of the AHE event or other important parameter for determining profound or sustained hypotension based on the MAP value, and clinicians can only react to absolute blood pressure threshold values for short-term blood pressure changes.

Therefore, there is a need to adaptively detect an AHE event of a patient by providing a personalized definition of AHE on MAP threshold value and integration with continuous hemodynamic waveform data to improve current models and develop new hypotension prediction models.

The present disclosure describes in various embodiments below a method, or an apparatus comprising a detection module configured to perform methods, for defining a patient-specific or personalized AHE on MAP waveform of a patient and detecting an AHE event of the patient based on a patient-specific or personalized MAP threshold value.

Advantageously, the present disclosure provides a personalized definition of hypotension based on a fluctuation of a MAP value to get an optimum target MAP threshold value which can prevent higher load of pressor. The present disclosure also provides a method and an apparatus comprising a multi-level prediction module configured to perform a method, for determining a potential occurrence of a subsequent AHE event as well as a level of severity of the AHE event or hypotension.

The present disclosure also describes in various embodiments below a method, and an apparatus comprising a multi-level prediction module configured to perform a method, for determining a short-term to long-term prediction of a subsequent AHE event of a patient based on the patient-specific or personalized AHE on MAP waveform of the patient. This may provide a prediction of hypotension in advance from 5 minutes to 2 hours, covering a short-term, intermediate term to long term prediction which can be beneficial in preemptive treatment for different care setting.

The present disclosure further provides a method and an apparatus for adaptively detecting an AHE event of a patient applicable in intraoperative care, intensive care and post-operative care setting (recovery ward) to monitor patient conditions. Such method and the apparatus utilizes hemodynamic data from routinely patient monitor unit hence they can be retrofitted to the existing healthcare units and settings, which in turn does not require any special and additional hardware device to acquire hemodynamic or other clinical data. Overall, the method and the apparatus for adaptively detecting an AHE event of a patient described in the present disclosure can solve the above-mentioned issues in the conventional AHE event detection systems.

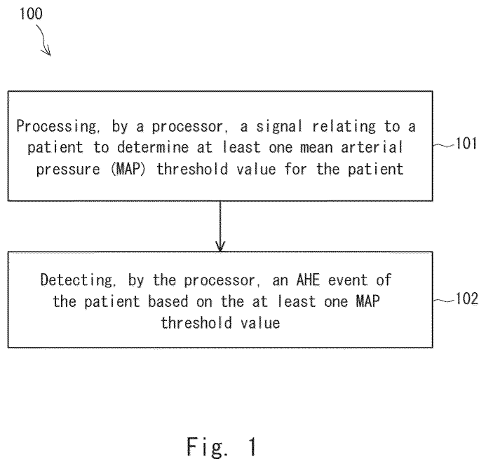

FIG. 1 shows a flow chart 100 illustrating a method for adaptively detecting an acute hypotension episode (AHE) event of a patient according to various embodiments of the present disclosure. In step 101, a step of processing, by a processor, a signal relating to a patient to determine at least one MAP threshold value for the patient is carried out. In step 102, a step of detecting, by the processor, an AHE event of the patient based on at least one MAP threshold value is carried out.

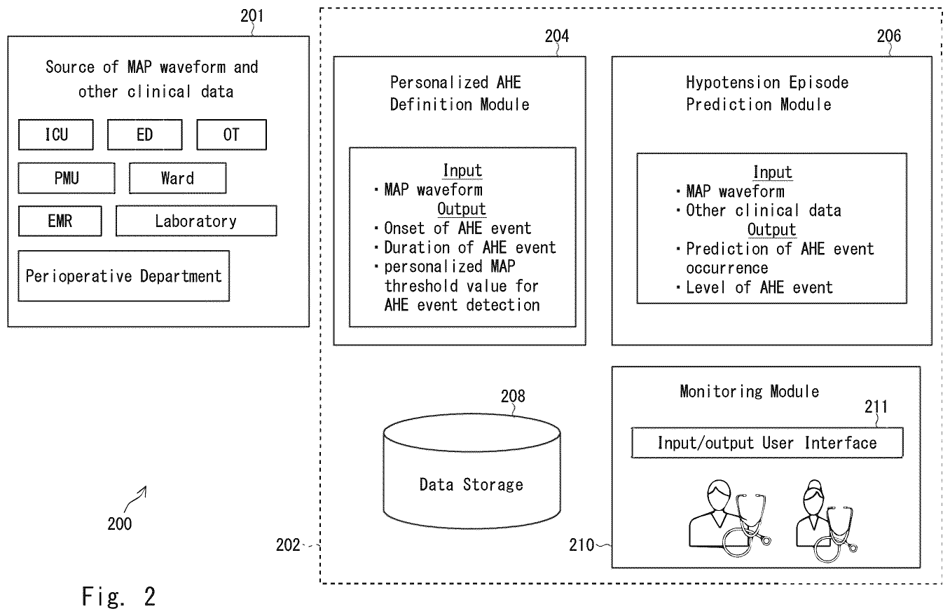

FIG. 2 shows a block diagram 200 illustrating an apparatus 202 for adaptively detecting an AHE event of a patient according to various embodiments of the present disclosure. In an implementation, the apparatus 202 may be generally described as a physical device comprising at least one processor and at least one memory including computer program code. The at least one memory and the computer program code are configured to, with the at least one processor, cause the physical device to perform the operations described in FIG. 1.

In an implementation, the apparatus 202 may receive signal such as MAP waveform and other clinical data from a source 201, for example from an equipment or a measurement conducted in intensive care unit (ICU), emergency department (ED), data input by occupational therapy (OT), patient monitoring unit (PMU), and/or data from perioperative departments, data from ambulance or medically equipped vehicle, ward, and/or electronic medical records (EMR). The apparatus 202 comprises a personalized AHE definition module 204 configured to determine at least one MAP threshold value for a patient and define personalize AHE based on the signal, a hypotension episode prediction module 206 configured to determine a prediction of a subsequent AHE event of the patient based on the signal.

In an implementation, the personalized AHE definition module 204 is configured to receive a MAP waveform of a patient as an input and output a detection of an AHE event of the patient including an onset of the AHE event, a duration of the AHE event and personalized MAP threshold value used for such detection based on the input. In an embodiment, the hypotension episode prediction module 206 is configured to receive a MAP waveform and other clinical data and output a prediction of an AHE event occurrence as well as a severity level of AHE event based on the input.

The apparatus 202 may further comprise a monitoring module 210 configured to display the signals from the source 201 and/or inputs and outputs of the personalized AHE definition module 204 and the hypotension episode prediction module 206 in a user interface 211 to an operator of the apparatus 202 such as a doctor, a medical staff or an analyst. The operator may also interact with the user interface 211 and input data into the apparatus 202 through the monitoring module 210. While it is shown in FIG. 2 that the monitoring module 210 is part of the apparatus 202, it should be appreciated that the monitoring module 210 may be standalone device or part of another device and is in communication with the apparatus 202 through a connection. Such connection may be wired, wireless (e.g., via NFC communication, Bluetooth, etc.) or over a network (e.g., the Internet).

The apparatus may comprise a data storage 208 accessible by the apparatus 202 for storing signals from the source 201 as well as inputs and outputs of the personalized AHE definition module 204 and the hypotension episode prediction module 206. While it is shown in FIG. 2 that the data storage is part of the apparatus 202, it should be appreciated that the data storage 208 may not form part of the apparatus 202 and is in communication with the apparatus through a connection. Such connection may be wired, wireless (e.g., via NFC communication, Bluetooth, etc.) or over a network (e.g., the Internet) or over a cloud server. In another implementation, each of the modules 204, 206, 210 and the signal source 201 may comprise its own data storage for storing its input/output data and signal.

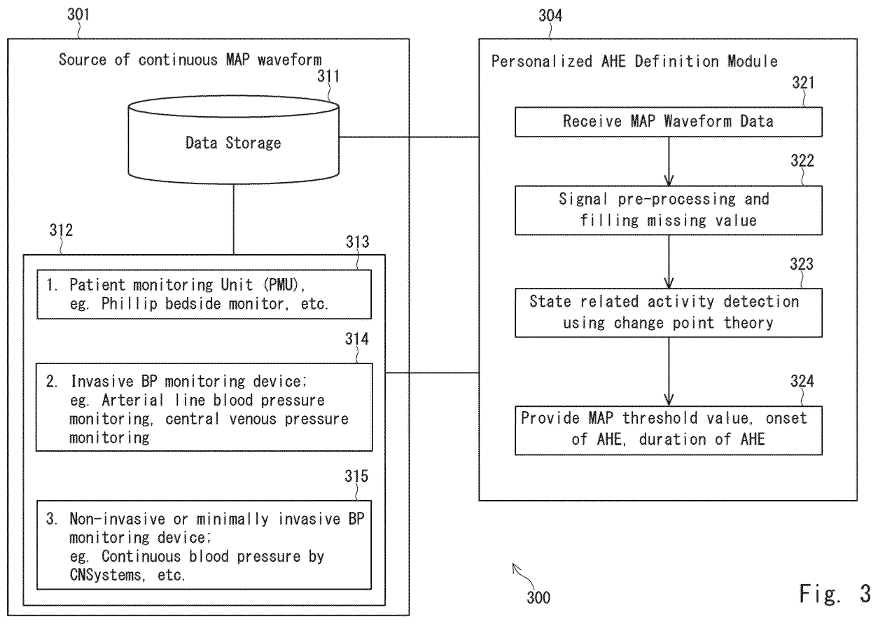

FIG. 3 shows a block diagram 300 illustrating a signal source 301 and a personalized AHE definition module 304 for determining a mean arterial pressure (MAP) threshold value for a patient according to an embodiment of the present disclosure.

The signal source 301 is a source of continuous MAP waveform of a patient measured from or conducted on the patient using devices comprising one or a combination of a patient monitoring unit 313 (e.g. a beside monitor of a patient), an invasive blood pressure monitoring device 314 (e.g. arterial line blood pressure monitoring device, central venous pressure monitoring device), and a non-invasive or minimally invasive blood pressure monitoring device 315 (e.g. continuous blood pressure monitoring system by CNSystems). The source 301 may further comprise a data storage configured to store the signal and measurement of the devices. In one implementation, the data storage 311 may be included in the personalized AHE definition module 304.

In an implementation, the personalized AHE definition module 304 may be generally described as part of a physical device comprising at least one processor and at least one memory including computer program code. The at least one memory and the computer program code are configured to, with the at least one processor, cause the physical device or the part to receive signal from the source 301 and perform operations in steps 321-324. In particular, the personalized AHE definition module 304 may be configured to carry out a step of receiving MAP waveform data relating to a patient directly from the signal source 301 and/or through the data storage 311 in step 321. The module 304 may be further configured to perform a step of processing the signal such as pre-processing the signal and filling missing value in the signal in step 322 and a step of stating related activity detection using change point theory in step 323, and a step of determining a MAP threshold value for the patient and providing a detection of an AHE event of the patient including an onset and a duration of the AHE event in step 324. The MAP threshold value for the patient and the data relating to the detected AHE onset and duration may be transmitted to the data storage 311 of the source 311.

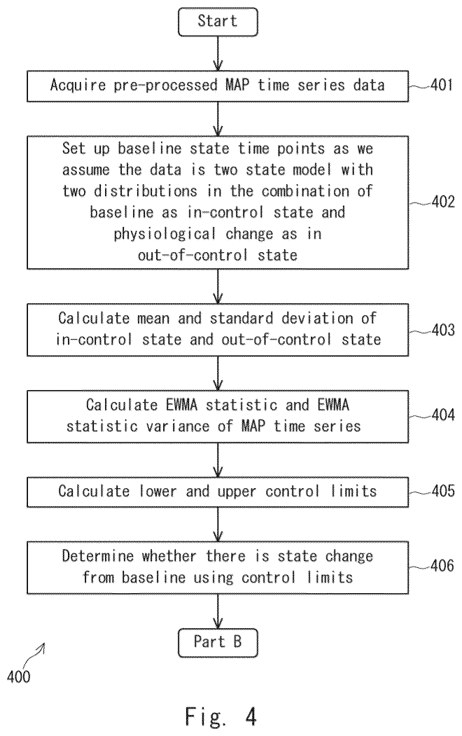

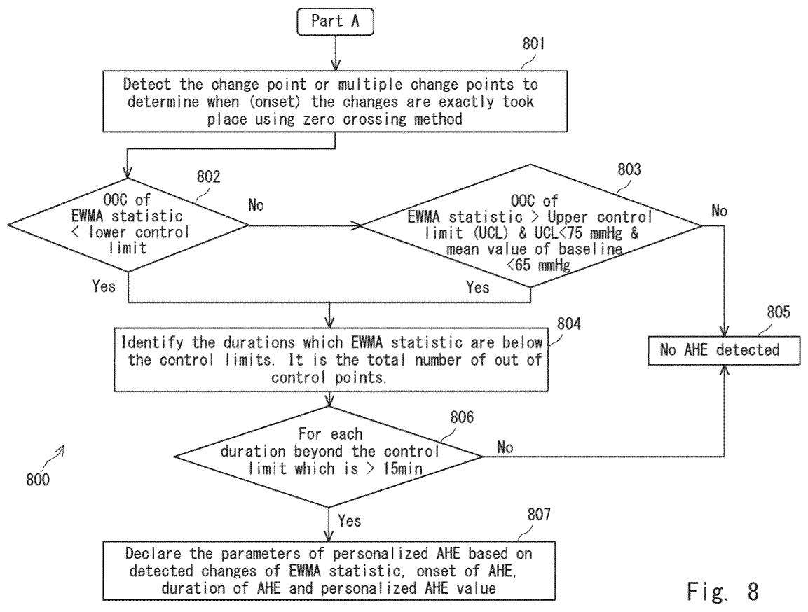

FIG. 4 shows a flow chart illustrating a part of a method for determining a MAP threshold value for a patient according to an embodiment of the present disclosure. In step 401, a step of acquiring pre-processed MAP waveform or time series data is carried out. In step 402, a step of setting up baseline state time points is carried out. In one example, the baseline state time points are set up by assuming that the data is a two-state model with two distributions in the combination of baseline as in-control state and physiological change as in out-of-control state. In step 403, a step of calculating mean and standard deviation of the in-control state and the out-of-control state is carried out. In step 404, a step of calculating exponentially weighted moving average (EWMA) statistic and EWMA statistic variance of the MAP time series data is carried out. In step 405, a step of calculating lower and upper control limits of the MAP time series data is carried out. In step 406, a step of determining whether there is a state change from the baseline using the calculated control limits is carried out. The process may then be directed to another part of the method shown in FIG. 8.

For setting up baseline sate time points in the pre-processed MAP waveform or time series data, a two-state model is first considered where the data is modelled as the combination of two normal distributions, one with mean θ₀ and (co)variance Σ, and the second with mean θ₁ and (co)variance Σ.

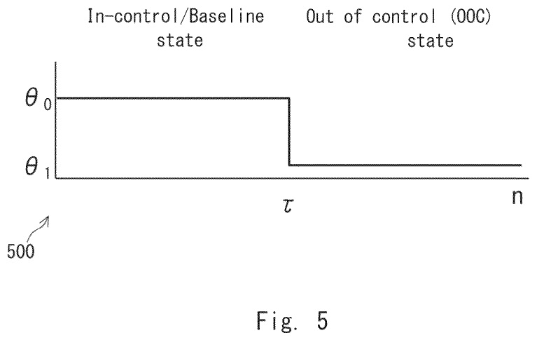

FIG. 5 shows a mean θ against time t graph illustrating two different states s of a two-state model used for determining a MAP threshold value for a patient according to an embodiment of the present disclosure. In this exemplary embodiment, in FIG. 5, two states, i.e. a in-control or baseline state and an out-of-control (OOC) state, are illustrated using measurements θ₀ and θ₁ at time t before τ (t=1, . . . , τ) and after τ (t=t+1, . . . , n) respectively: while the current state value sₜ can be summarized using equation (2):

$${s}_{t} = \{ \begin{matrix}{θ}_{0} & for t = 1 , … , τ \\ {θ}_{1} & for t = τ + 1 , … , n\end{matrix} \qquad equation ( 2 )$$

### The Mean Observation

\vec{x}
and current observation xₜ can be calculated using the following equations:

$$\vec{x} = {( {x}_{1} , {x}_{2} , {x}_{3} , … , {x}_{n} )}^{T} \qquad equation ( 3 )$$

$${x}_{t} = {s}_{t} + {ε}_{t} \qquad equation ( 4 )$$

where εₜ˜N(0,Σ) follows a mean zero normal distribution with covariance Σ.

EWMA statistic zₜ, which is a smoothed version of MAP waveform or time series data can be computed using equation (5):

$${z}_{t} = λ {x}_{t} + ( 1 - λ ) {z}_{i - 1} for t = 1 , … , n \qquad equation ( 5 )$$

Each value of zₜ is a weighted average of the current observation xₜ and the previous value of the EWMA statistic. For simplicity, the starting value z₀ is set to equal to the baseline measurement θ₀ (at t=0) Hence, equation (5) can also be written as follows:

$${z}_{t} = λ {\sum }_{j = 0}^{t - 1} {( 1 - λ )}^{j} {x}_{i - j} + {( 1 - λ )}^{t} {θ}_{0} for t = 1 , … , n \qquad equation ( 6 )$$

where 0<λ<1 is a constant smoothing parameter chosen by an analyst or a user of the apparatus of the present disclosure for pre-processing the MAP waveform or time series data. When λ is small, a more smoothing effect on the data is resulted; and when λ is large, a less smoothing effect on the data is resulted.

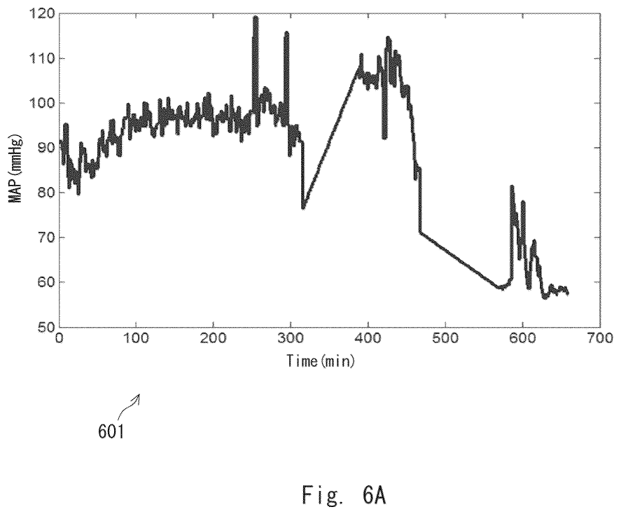

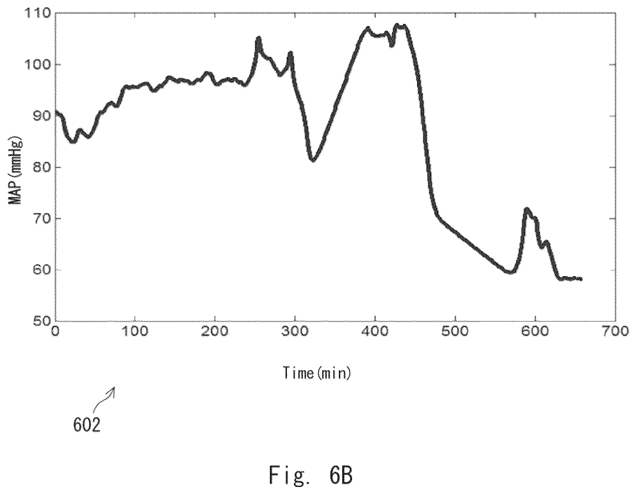

FIGS. 6A and 6B show a MAP waveform 601 and a smoothened MAP waveform 602 respectively according to an embodiment of the present disclosure. The smoothed MAP waveform 602 may be resulted from the processing of the MAP waveform 601 through EWMA statistic and equations (5) and (6).

EWMA statistic variance var(zₜ) is a variance of the EQMA statistic zₜ, which under white noise can be calculated based on equation (7) below:

$$var ( {z}_{t} ) = {σ}^{2} \frac{λ}{2 - λ} ( 1 - {( - λ )}^{2 t} ) \qquad equation ( 7 )$$

The upper and lower control limits (UCL/LCL) are defined based on the EWMA statistic zₜ deviations from baseline using the following equation:

$${θ}_{0} \pm {t}^{*} \times \sqrt{var ( {z}_{t} )} \qquad equation ( 8 )$$

where t* is a critical value from t-distribution.

Using two-state model, if zₜ at any time exceeds/below the control limits calculated from equation (8), it is determined that there is a fluctuation from baseline and the process is deemed to have changed states from its baseline region.

If a change state or fluctuation is detected, a step of calculating when exactly the change took place is carried out to estimate the unknown parameter τ, i.e. the change point (time point) at which the state change took place. “Zero-crossing” method is one of several methods that can be utilized for estimating τ. Based on “zero-crossing” method, the last time point at which the process crosses θ₀ is the estimate of τ.

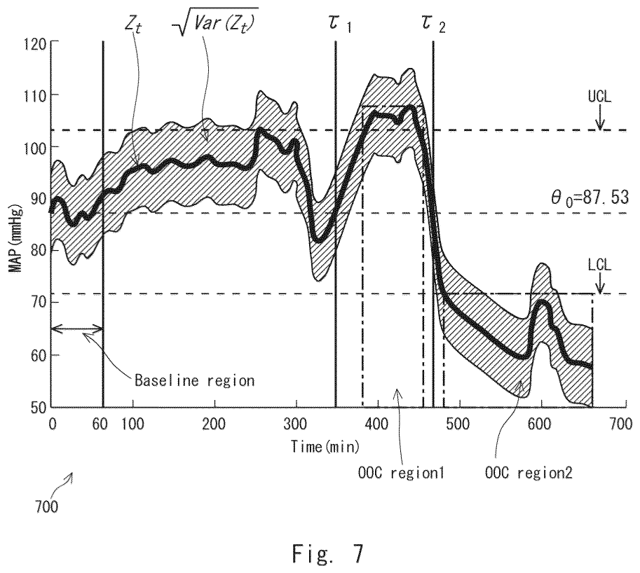

FIG. 7 shows a processed MAP waveform 700 and personalized AHE parameters according to an embodiment of the present disclosure. In one example, the processed MAP waveform is derived using two-state model and equations (2)-(8) shown above, and the derivatives such as baseline region, baseline measurement θ₀, EWMA statistic zₜ (i.e. processed MAP waveform), EWMA statistic variance var(zₜ) (i.e. variance of the MAP waveform), UCL, LCL, out-of-control (OOC) region and estimated t are obtained in the processing, as indicated in FIG. 7.

In this embodiment, the first 60 minutes of the processed MAP waveform 700 is determined as a baseline region of the MAP waveform 700 and the baseline, i.e. the baseline measurement θ₀ in this case, is the mean of the MAP values of the baseline region of 87.53 mmHg.

The UCL and the LCL are calculated based on the baseline and the fluctuation of the MAP waveform 700. In this case, the fluctuation is determined based on the EWMA statistic variance var(zₜ) of the MAP waveform 700 as indicated in the grey area around the waveform 700. OOC regions are set at a region where the MAP values are higher than the UCL or lower than the LCL such as OOC region 1 and region 2 respectively.

Each estimated time point at which the state shift from a baseline state to an OCC state can be determined using “zero-crossing” method by referring to the last time point at which the processed MAP waveform crossed θ₀. In this embodiment, the estimated change points at which a change to OOC region 1 and OOC region 2 took place are τ₁ and τ₂, or roughly at 340 min and 480 min point, respectively.

FIG. 8 shows a flow chart illustrating another part of a method for determining a MAP threshold value for a patient according to an embodiment of the present disclosure. In one example, after a state change from in-control state to out-of-control state is detected in step 406, a step 801 of detecting the change point or multiple change points to determine an onset of the change(s) are exactly took place using zero crossing method. In step 802, a step of determining if the detected OOC region(s) of the processed signal (EWMA statistic) has a value lower than the LCL. If the value is lower than the LCL, step 804 is carried out; otherwise step 803 is carried out. In step 803, a step of determining if the detected OOC region(s) of the processed signal (EWMA statistic) has a value higher than the UCL, the UCL is lower than a pre-configured threshold value, in this case the pre-configured threshold value is 75 mmHg, and a mean value of the baseline is lower than a pre-configured threshold value, in this case the pre-configured threshold value is 65 mmHg. If all three conditions are met, step 804 is carried out: otherwise step 805 is carried out.

In step 804, a step of identifying each of the durations of the processed signal (EWMA statistic) beyond the control limit, i.e. OOC regions, is longer than a pre-configured duration, in this case the pre-configured duration is 15 minutes (step 806). If the duration is longer than the pre-configured duration, step 807 is carried out; otherwise, step 805 is carried out.

In step 805, it is determined that no personalized AHE is detected. In step 807, a step of declaring parameters of personalized AHE based on detected changes of EWMA statistic, onset of the AHE, duration of the AHE and personalized AHE value, i.e. MAP threshold value defined for the patient. Subsequently, detections of AHE events of the patient are carried out based on the personalized AHE/MAP threshold value.

Various embodiments below illustrate MAP waveform assessment, personalized AHE definition and AHE event detections using the steps in the flow chart 800 in FIG. 8 and its accompanying description above. However, it is appreciated by person skilled in relevant art(s) that it is just one of many implementations. Different type of signals (e.g. HR and SPO₂) and steps with different criteria may be used to determine personalized AHE definition for different patients.

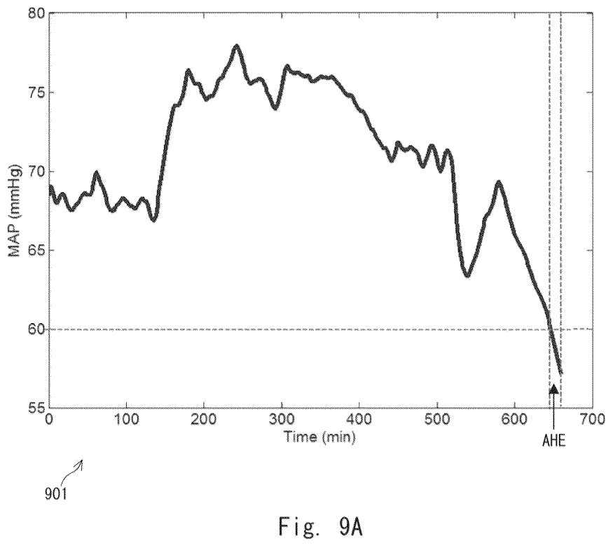

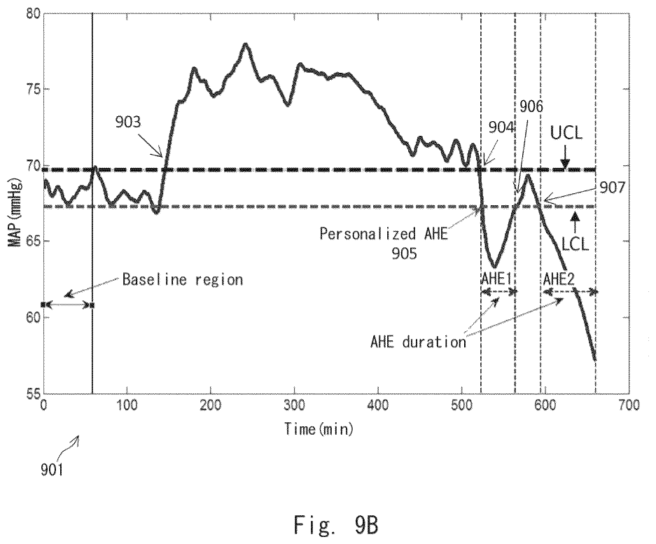

FIG. 9A shows a processed MAP waveform 901 with AHE detected using conventional single threshold method and FIG. 9B shows personalized AHE parameters of the processed MAP waveform 901 according an embodiment of the present disclosure. In FIG. 9A, an AHE is detected when the MAP data falls lower than a fixed MAP threshold value of 60 mmHg. FIG. 9B depicts a baseline region in the first 60 minutes of the same processed MAP waveform 901 and UCL/LCL calculated based on the baseline and fluctuations of the processed MAP waveform 901. It is noted that the UCL is lower than a pre-configured threshold value of 75 mmHg and the mean value of baseline is higher than a pre-configured threshold value of 65 mmHg.

A few state changing points are detected such as points 903-907. In particular, point 905 meets the criteria of having a MAP value lower than LCL and a duration of the state lasted longer than a pre-configured threshold duration of 15 minutes. As such, a personalized AHE for the patient is defined at point 905 at MAP of 67.41 mmHg (LCL). Two AHE events are detected in regions between points 905-906 and points 906-907 respectively based on the personalized AHE, and their AHE parameters such as onsets (points 905, 907) and durations of the AHE events are declared and recorded. For this embodiment, personalized AHE was detected earlier than the convention single threshold. This may provide more time for clinicians to administer the patient for AHE.

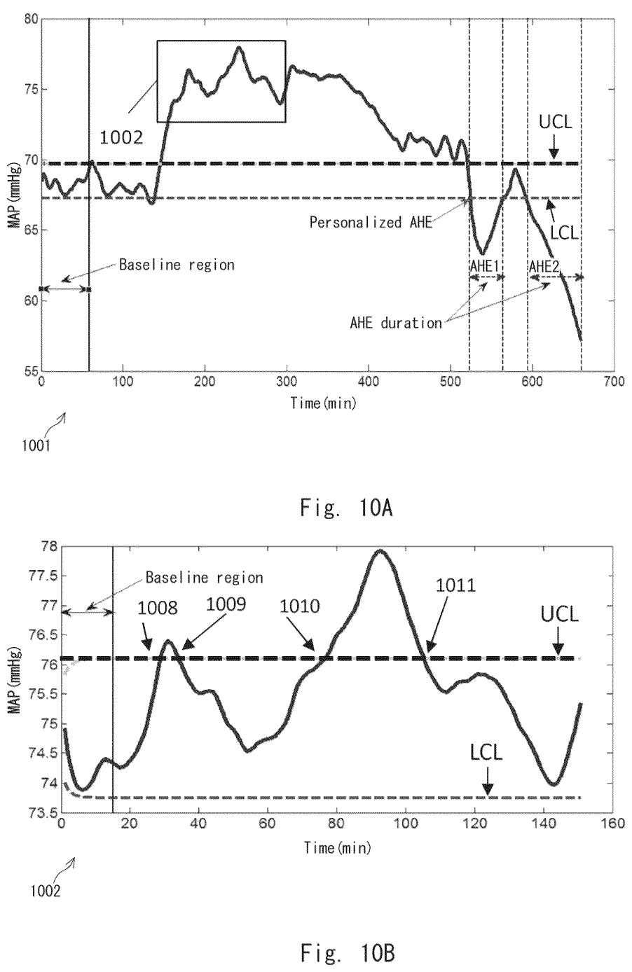

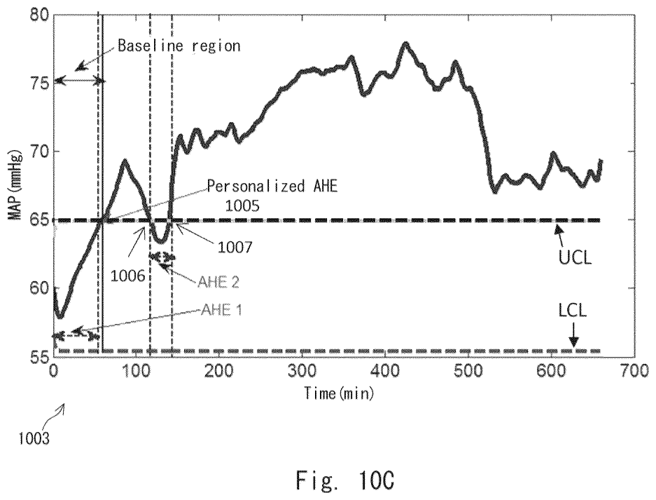

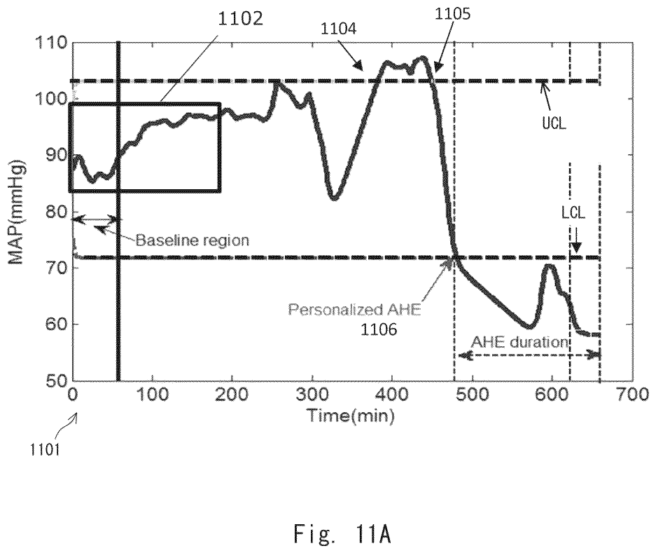

FIG. 10A shows a processed MAP waveform 1001 of a patient and personalized AHE parameters which are identical to FIG. 9B. FIG. 10B shows a subsample 1002 of the processed MAP waveform 1001. A new baseline region in the first 15 minutes of that portion is determined and UCL/LCL is calculated based on the new baseline and fluctuations of MAP values within that subsample. There is no state change from in-control state to OOC state beyond LCL. A few state changing points between in-control state and OOC state beyond UCL such as points 1008-1011 are detected in the waveform 1002. However, it is noted that the UCL is higher than a pre-configured threshold value of 75 mmHg and the mean value of baseline is higher than a pre-configured threshold value of 65 mmHg. None of state changing points meets the criteria set in FIG. 8. As such, no personalized AHE was detected.

FIG. 10C shows personalized AHE parameters of a processed MAP waveform 1003 relating to a patient with high volume and blood lost due to an accident before arriving at accident and emergency department of a hospital and already have hypotension episode, e.g. AHE 1. FIG. 10C depicts a baseline region in the first 60 minutes of the processed MAP waveform 1003 and UCL/LCL calculated based on the baseline and fluctuations of the processed MAP waveform 1003. It is noted that the UCL is lower than a pre-configured threshold value of 75 mmHg and the mean value of baseline is lower than a pre-configured threshold value of 65 mmHg.

Three state changing points are detected such as points 1005-1007 in the MAP waveform 1003. In particular, point 1005 meets the criteria of having a MAP value higher than UCL, mean MAP of baseline less than 65 mmHg and a duration of the state lasted longer than a pre-configured threshold duration of 15 minutes. As such, a personalized AHE for the patient is defined at point 1005 at MAP of 64.9 mmHg (LCL). An AHE event are detected in regions between points 1006-1007 based on the personalized AHE, and their AHE parameters such as onsets (point 1006) and durations of the AHE events are declared and recorded. For this embodiment, personalized AHE was detected earlier than another hypotension episode, e.g. AHE 2.

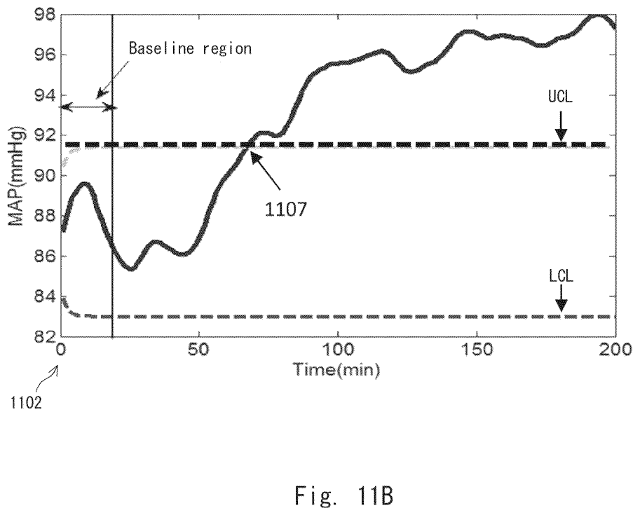

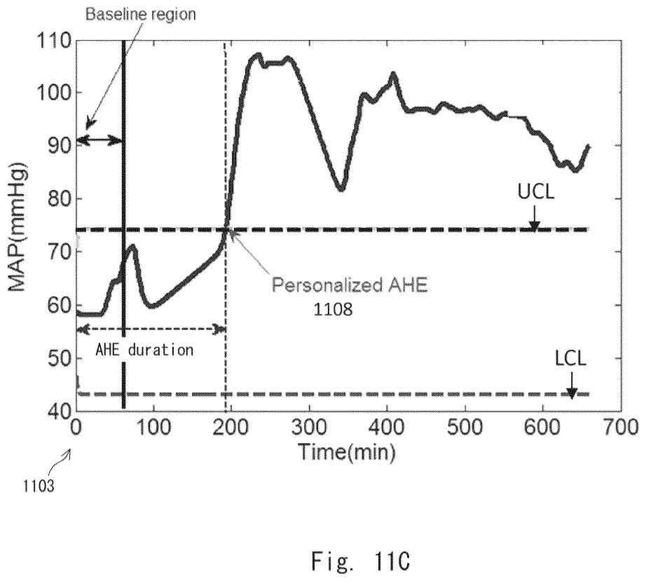

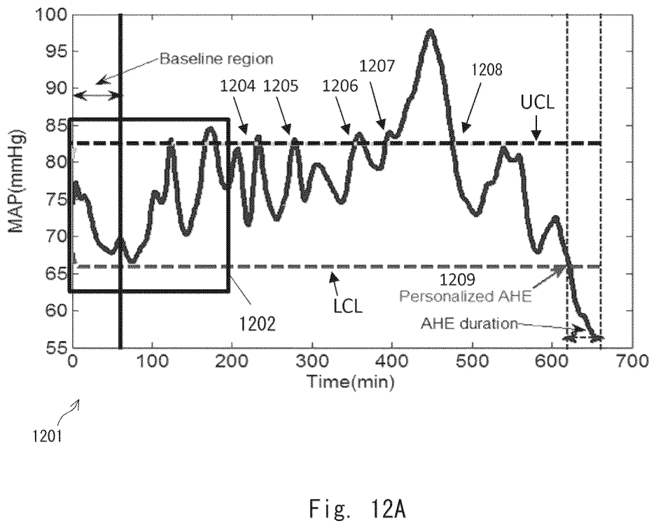

FIG. 11A shows personalized AHE parameters of a processed MAP waveform 1101 of a patient according to an embodiment. FIG. 11A depicts a baseline region in the first 60 minutes of the processed MAP waveform 1101 and UCL/LCL calculated based on the baseline and fluctuations of the processed MAP waveform 1101. It is noted that the UCL is higher than a pre-configured threshold value of 75 mmHg and the mean value of baseline is higher than a pre-configured threshold value of 65 mmHg.

Three state changing points are detected such as points 1104-1106 in the MAP waveform 1101. In particular, point 1106 meets the criteria of having a MAP value lower than LCL and a duration longer than a pre-configured threshold duration of 15 minutes. As such, a personalized AHE for the patient is defined at point 1106 at MAP of 71.63 mmHg (LCL).

FIG. 11B shows a subsample 1102 of the processed MAP waveform 1101. A new baseline region in the first 15 minutes of that portion is determined and UCL/LCL is calculated based on the new baseline and fluctuations of MAP values within that subsample. It is noted that the UCL is higher than a pre-configured threshold value of 75 mmHg and the mean value of baseline is higher than a pre-configured threshold value of 65 mmHg. A state changing point from in-control state to OOC state beyond UCL is detected at point 1107 which does not meet the criteria. No state change from in-control state to OOC state beyond LCL is detected. As such, no personalized AHE was detected.

FIG. 11C shows personalized AHE parameters of a processed MAP waveform 1103 relating to a patient with high volume and blood lost due to an accident before arriving at accident and emergency department of a hospital and already have hypotension episode. FIG. 11C depicts a baseline region in the first 60 minutes of the processed MAP waveform 1103 and UCL/LCL calculated based on the baseline and fluctuations of the processed MAP waveform 1103. It is noted that the UCL is lower than a pre-configured threshold value of 75 mmHg and the mean value of baseline is lower than a pre-configured threshold value of 65 mmHg.

A state changing point from in-control state to OOC state beyond UCL is detected at point 1108 in the MAP waveform 1003. In particular, point 1108 meets the criteria of having a MAP value higher than UCL, mean MAP of baseline is less than 65 mmHg and a duration the state lasted longer than a pre-configured threshold duration of 15 minutes. As such, a personalized AHE for the patient is defined at point 1108 at MAP of 64.9 mmHg (UCL). For this embodiment, personalized AHE was detected at an earlier time point than conventional methods.

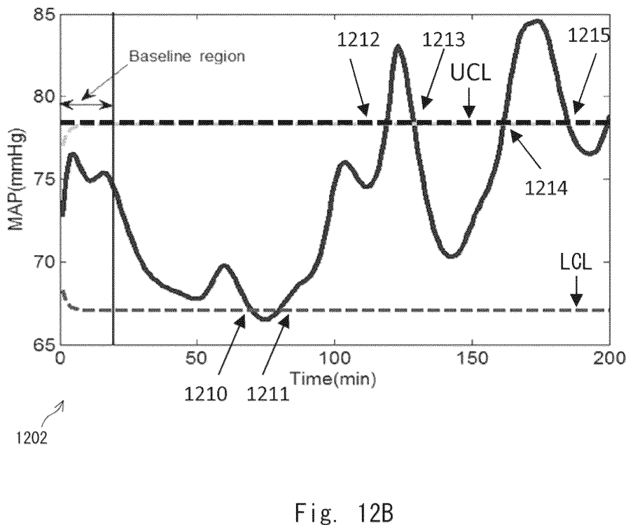

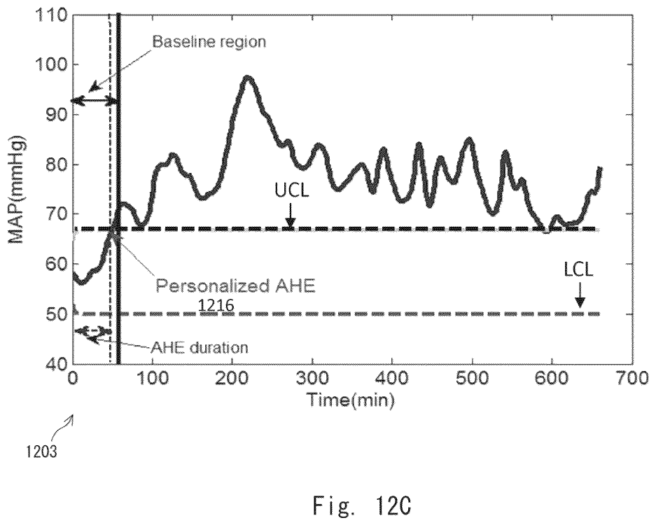

FIG. 12A shows personalized AHE parameters of a processed MAP waveform 1201 of a patient according to an embodiment. FIG. 12A depicts a baseline region in the first 60 minutes of the processed MAP waveform 1201 and UCL/LCL calculated based on the baseline and fluctuations of the processed MAP waveform 1201. It is noted that the UCL is higher than a pre-configured threshold value of 75 mmHg and the mean value of baseline is higher than a pre-configured threshold value of 65 mmHg.

A few state changing points such as points 1204-1209 are detected in the MAP waveform 1201. In particular, point 1209 meets the criteria of having a MAP value lower than LCL and a duration longer than a pre-configured threshold duration of 15 minutes. As such, a personalized AHE for the patient is defined at point 1106 at MAP of 65.65 mmHg (LCL).

FIG. 12B shows a subsample 1202 of the processed MAP waveform 1201. A new baseline region in the first 15 minutes of that portion is determined and UCL/LCL is calculated based on the new baseline and fluctuations of MAP values within that subsample. It is noted that the UCL is higher than a pre-configured threshold value of 75 mmHg and the mean value of baseline is higher than a pre-configured threshold value of 65 mmHg. Six state changing points 1210-1215 are detected, but none of them meets the criteria set in FIG. 8. For example, even point 1210 has a MAP value lower than LCL, but it does not last longer than a pre-configured duration of 15 minutes. As such, no personalized AHE was detected from FIG. 12B.

FIG. 12C shows personalized AHE parameters of a processed MAP waveform 1203 relating to a patient with high volume and blood lost due to an accident before arriving at accident and emergency department of a hospital and already have hypotension episode. FIG. 12C depicts a baseline region in the first 60 minutes of the processed MAP waveform 1203 and UCL/LCL calculated based on the baseline and fluctuations of the processed MAP waveform 1203. It is noted that the UCL is lower than a pre-configured threshold value of 75 mmHg and the mean value of baseline is lower than a pre-configured threshold value of 65 mmHg.

A noticeable state changing point from in-control state to OOC state beyond UCL is detected at point 1216 in the MAP waveform 1203. In particular, point 1216 meets the criteria of having a MAP value higher than UCL and a duration the state lasted longer than a pre-configured threshold duration of 15 minutes. As such, a personalized AHE for the patient is defined at point 1216 at MAP of 65.65 mmHg (UCL). For this embodiment, personalized AHE was detected at an earlier time point than conventional methods.

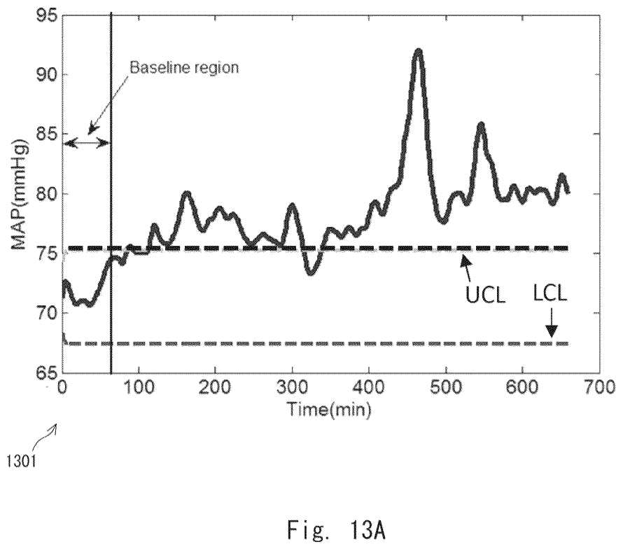

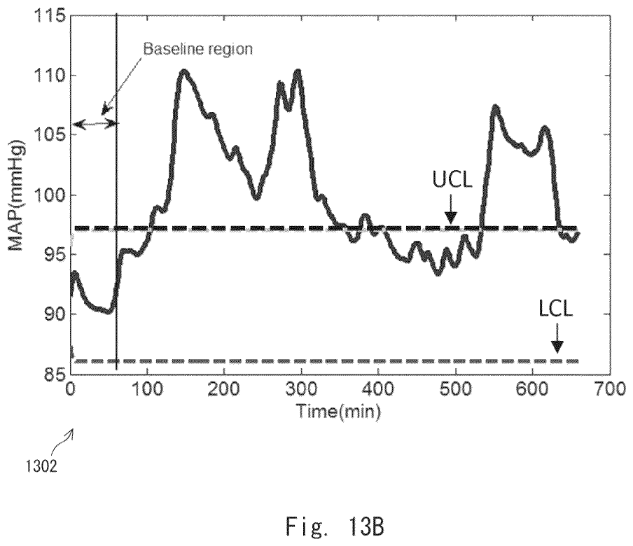

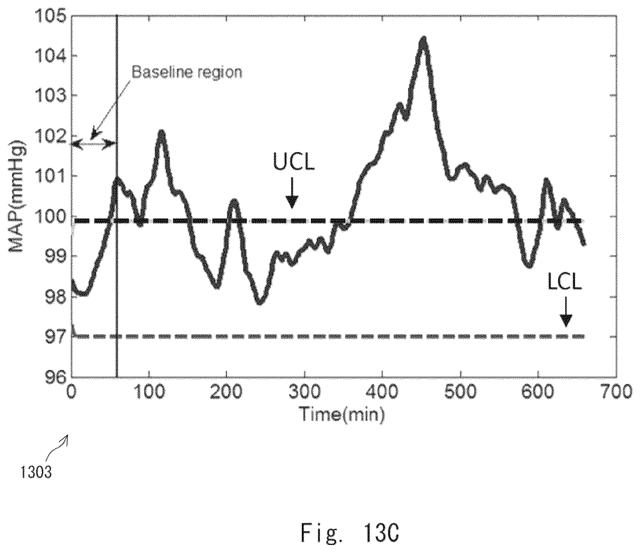

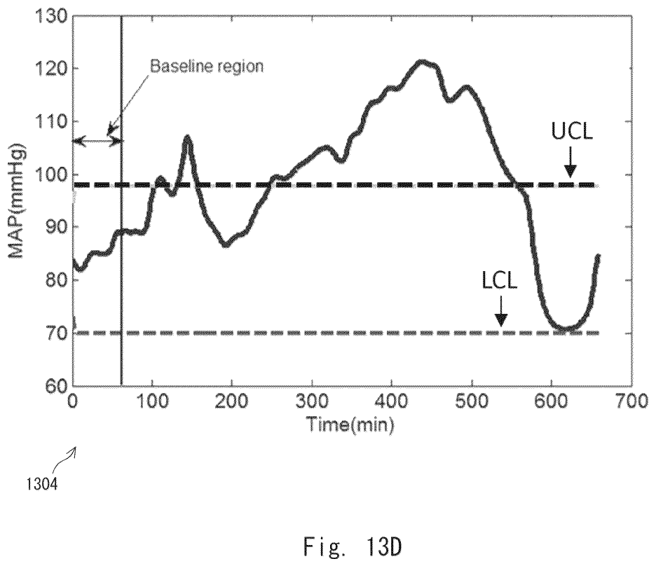

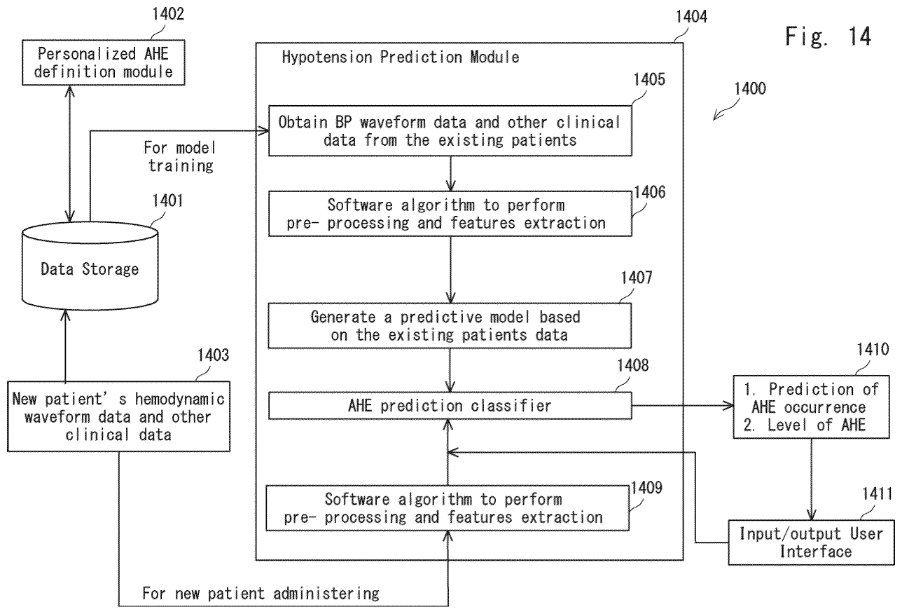

FIGS. 13A-13D show four example MAP waveforms with patients with no AHE event. AHE was not detected in all four example MAP waveforms based on the steps of the flow chart 800 of FIG. 8 and its accompanying description.

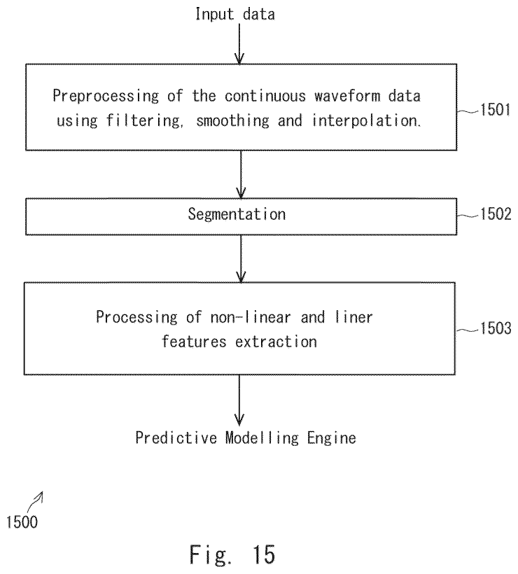

FIG. 14 shows a block diagram of a hypotension prediction module 1404 for adaptively detecting and predicting an AHE event of a patient according to an embodiment of the present disclosure. In an implementation, the hypotension prediction module 1404 may be generally described as part of a physical device comprising at least one processor and at least one memory including computer program code. The at least one memory and the computer program code are configured to, with the at least one processor, cause the physical device or the part to perform operations steps 1405-1407 for model training and steps 1408-1409 for new patient administering based on the trained model.

A data storage 1401 is configured to receive signals relating to new patients such as waveform data and other clinical data 1403 and is accessible by the hypotension episode prediction module 1401. Additionally, such data storage 1401 may also be shared between the hypotension episode prediction module and a personalized AHE definition module 1402. The data stored therein can be used by the personalized AHE definition module to determine a personalized AHE and detect an AHE event in the data. The personalized AHE and other parameters may be recorded in the data storage and optionally used by the hypotension prediction module 1404 to generate its output.

For model training, the hypotension prediction module 1404 is configured to carry out a step of obtaining signal such as blood pressure waveform and other clinical data relating to a plurality of patients (e.g. existing patients) in step 1405, a step of running software algorithm to perform pre-processing and feature extraction of the waveform data and clinical data relating to the existing patients in step 1406 and a step of generating a predictive model based on the extracted features and corresponding feature data set (hereinafter referred to “training data set” or “testing data set”) of the waveform data and clinical data in step 1407.

For new patient administering, for example after a new patient for which a personalized AHE has just been determined, the hypotension prediction module is configured to carry out a step of running software algorithm to perform pre-processing and feature extraction of received waveform data and other clinical data 1403 relating the new patient in step 1409, and then a step of running an AHE prediction classifier where the extracted features and feature data (hereinafter referred to as “new data set”) from the new patient's signal 1403 is classified using the predictive model generated in step 1407 and determining a prediction of a subsequent AHE event of the new patient.

A prediction of an occurrence of a subsequent AHE event of the new patient as well as its severity level of the AHE event may be determined, as indicated in block 1410.

The hypotension prediction module 1404 may be configured to display the new patient's signal 1403, inputs and outputs of the hypotension prediction module 1404 in a user interface 1411 to an operator of the apparatus 202 such as a doctor, a medical staff or an analyst. The operator may also interact with the user interface 1411 and input data into the hypotension prediction module 1404 or other module in connection with the module 1404. In one implementation, the user interface 1411 is configured to be displayed on a monitoring module (not shown) integrated into or separated from the hypotension prediction module 1404 but is in communication with the hypotension prediction model 1404 through a connection. Such connection may be wired, wireless (e.g., via NFC communication, Bluetooth, etc.), over a network (e.g., the Internet) or another device to which both modules are in communication with.

In various embodiments of the present disclosure, the term hypotension prediction module may be described as predictive modelling engine and are used interchangeably.

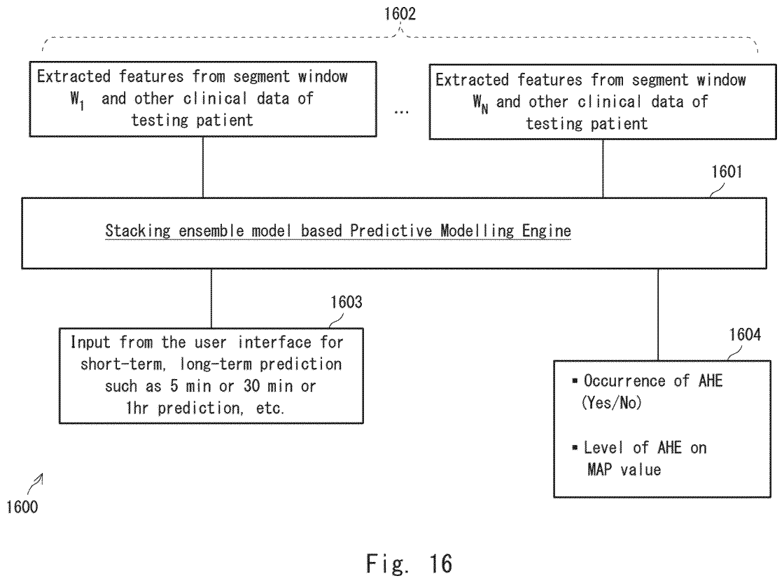

FIG. 15 shows a flow chart illustrating a part of a method of pre-processing a signal and extracting features of the signal for determining a prediction of an AHE event of a patient according to an embodiment of the present disclosure.

In one example, high fidelity hemodynamic waveform data and others clinical data such as HR, SBP, DBP, MAP, PP, SPO2%, and etc. are received. For example, at least MAP waveform of existing patients in which their waveform data are stored in database or cloud. These stored patients data had or did not have experience of AHE during their stay in ICUs or during operation for surgery. The others waveform data and clinical data may also be received. The hemodynamic waveform data is sampled with 125 Hz frequency or other sampling rate such as once per minute or once per second.

After receiving the input data, such as hemodynamic data and other clinical data relating to a plurality of testing patients used for model training or that of a new patient for prediction of AHE event and administering, pre-processing of the input data (e.g. continuous waveform data) is carried out using filtering, smoothing and interpolation in step 1501. In one example, noisy input data was pre-processed by removing baseline, ejecting extreme data values, smoothing and filling missing value by linear interpolation.

The pre-processed data will then go through a segmentation process in step 1502. In one example, sliding window method with window size of 10 s without overlapping is applied.

Processing of non-linear and linear features extraction will then be performed on the segmented data in step 1503. In one example, Time dependent features are extracted from the segmented waveform data. These features are; maximum power, mean power, min power, maximum frequency, mean frequency, median frequency, minimum frequency, maximum of segmented data, mean of segmented data, minimum of segmented data, variance of segmented data, skewness of segmented data, kurtosis of segmented data, RMS value of segmented data, 1st autoregressive of segmented data, slope sign change of data of segmented data, zero crossing of segmented data, autocorrelation of lag 1 to lag 5, 0% quantile, 25% quantile, 50% quantile, 75% quantile, 100% quantile.

Subsequently, the extracted features and corresponding features data are sent to predictive modelling engine, for example predictive modelling engine 1601 in FIG. 16, for further processing.

FIG. 16 shows a block diagram 1600 illustrating inputs and outputs of a stacking ensemble model based predictive modelling engine 1601 used for adaptive detecting and predicting an AHE event of a patient according to an embodiment of the present disclosure.

The stacking ensemble model based predictive modelling engine 1601 may receive extracted features from segment window W₁ and other clinical data relating to a plurality of patients (e.g. testing patients or existing patients), as indicated in 1602. The predictive modelling engine 1601 may also receive input from a user interface such as a time frame or decision time window where an occurrence of an AHE event of a new patient within the time frame is predicted. The time frame may refer to a short term or time window such as 3 to 30 min, or a long term or time window such as 3 hours to 3 days, and the predictive modelling engine 1601 will provide a prediction of an AHE event to occur within the time frame or decision time window accordingly. With the inputs 1602, 1603, the predictive modelling engine 1601 will determine a prediction of an occurrence of an AHE event of the new patient as well as a severity level of the AHE event, and output the prediction to the user interface.

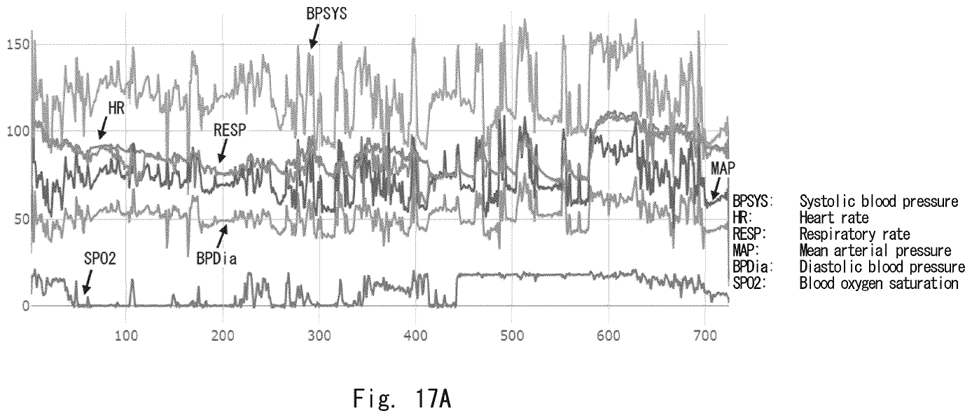

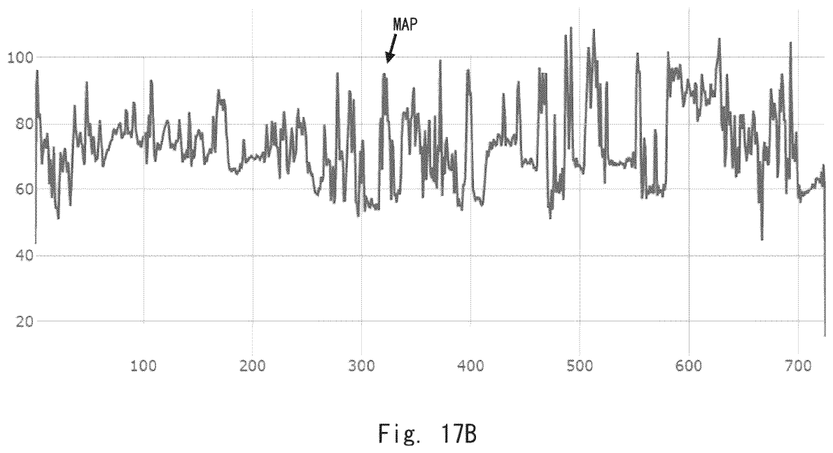

FIG. 17A shows various input data used for adaptively detecting and predicting an AHE event of a patient according to various embodiments of the present disclosure. FIG. 17B shows a MAP waveform used as an input for adaptively detecting and predicting an AHE event of a patient according to an embodiment of the present disclosure. In this embodiment, the MAP waveform data relates to a patient with a history of AHE.

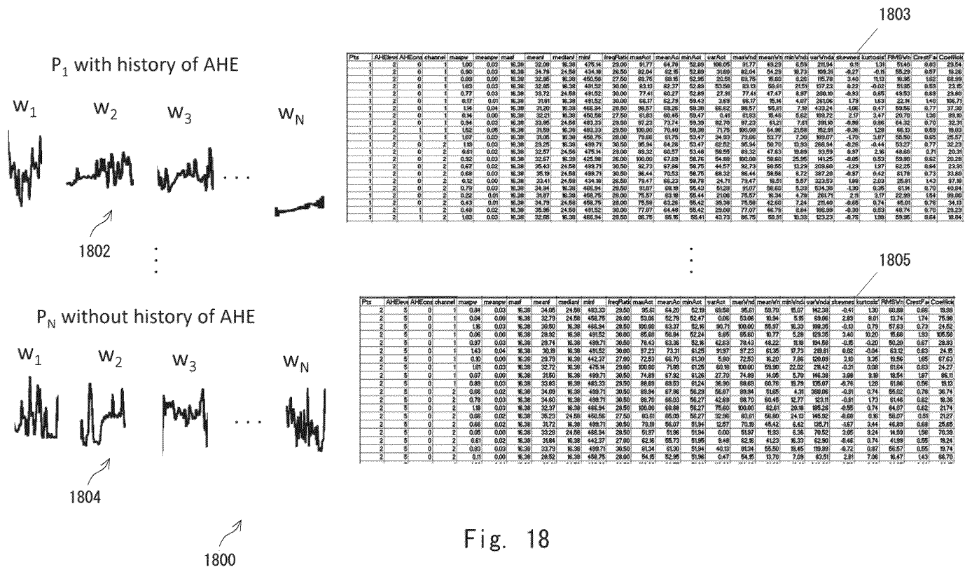

FIG. 18 shows features extracted from signals relating to a plurality of patients used for model training according an embodiment of the present disclosure. Two signals 1802, 1804 of a plurality of signals are shown, one of which relating to a patient P₁ with history of AHE and another one relating to a patient P_{N} without a history of AHE. Each signal will be pre-processed and segmented according to a plurality of segment windows W₁-W_{N}, and features of signal in each of the segment windows W₁-W_{N} and corresponding feature data are extracted. The data corresponding to the extracted features from the signals relating to P₁ and P_{N} are illustrated in tables 1803, 1805.

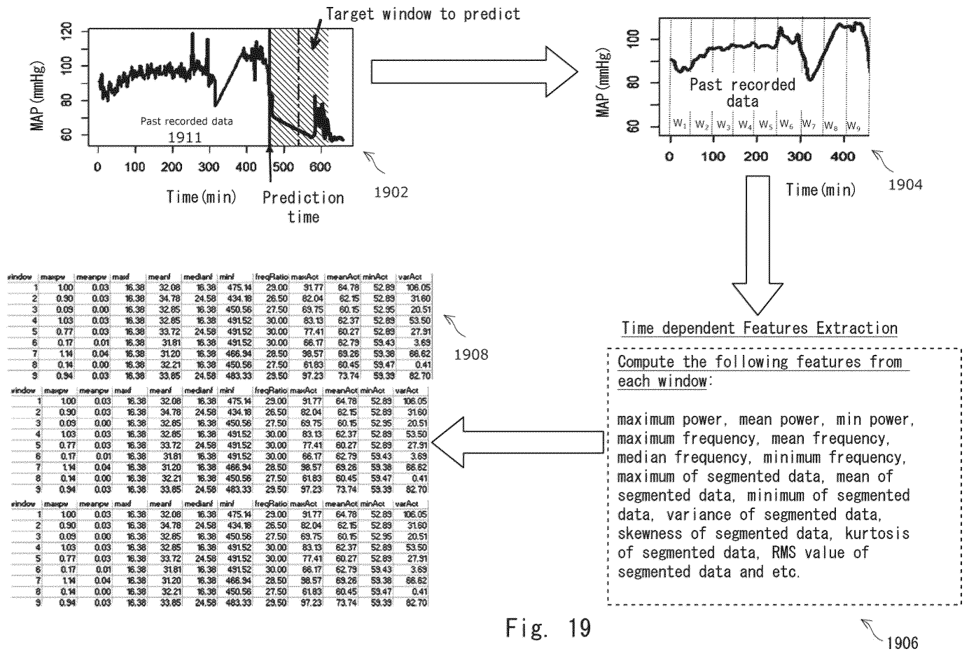

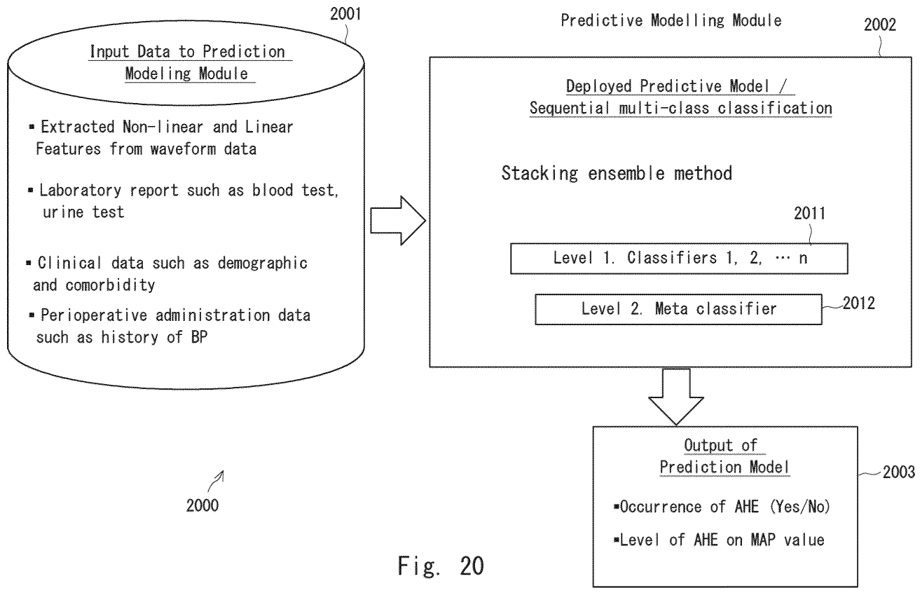

FIG. 19 shows a diagram illustrating a process of extracting at least one feature in a signal used for adaptively detecting and predicting an AHE event of a patient according an embodiment of the present disclosure. In this embodiment, an input of a signal relating to a patient is received. The input may further comprise past recorded data 1911 of the patient as well as target time frame 1912 to predict an AHE event of the patient. The past recorded data may be segmented according to a plurality of segment windows W₁-W₉, and features from each of the segment windows W₁-W₉ are computed to generate feature data as shown in table 1908. Such feature data will then be used for model training and/or AHE event prediction.

FIG. 20 shows a block diagram illustrating an apparatus 2000 for detecting and predicting an AHE event of a patient according to an embodiment of the present disclosure. The apparatus 2000 configured to receive time dependent features/feature data from feature extraction module (not shown) or algorithm, such feature including extracted non-linear and linear features waveform data, feature data including data from laboratory report such as blood test, urine test, clinical data such as demographic and comorbidity data, perioperative administration data such as history of BP, frailty score. The input data may also include an input of a time frame (short term or long term) within which where a prediction of an occurrence of an AHE event of a new patient is to be determined.

The apparatus 2000 is further configured to generate a deployed predictive model based upon at least one of these features. Particularly, the prediction model generates a prediction of AHE occurrence by applying the stacking ensemble method, and in the generation of the prediction, sequential multiple classification (or multi-class classification) for multilevel of AHE is applied to gain high performance and to avoid over fitting of the learning and model training. For example, at the first level, two or more predictions based on at least one extracted feature are determined using multiple base classifiers 2022 (classifiers 1, 2, . . . n) such as Random Forest, Gradient Boosting, Naive Bayes, generalized linear model. At the same time, this approach is not limited to our classification algorithm, but adaptive to various designs of classifiers such as SVM, KNN, neural networks, etc.

At the second level, a comparison of the two or more predictions is carried out to determine an optimal prediction (based on either one of the predictions or a combination of them) or an optimal prediction model among the multiple base classifiers (either one of a combination of them), that could detect, at best, correctly most subsequent AHE events of the testing patients. Such optimal prediction model is then applied to determine a prediction of a subsequent AHE of the new patient. In one example, majority voting approach is used as a meta classifier and it is not limited to our classification algorithm, but adaptive to other designs of classifier such as logistic regression, neural network, Xgboost, etc.

The apparatus is further configured to generate a prediction of an occurrence of a subsequent AHE event of a patient, for example, a yes/no output or a probability of occurrence (in %), and the onset of AHE within predefined time frame. In addition, a MAP value or a MAP value range of the subsequent AHE event is also determined. This MAP value or value range corresponds to a severity level of the subsequent AHE event. For example, the apparatus 2000 is further configured to generate a severity level of the subsequent AHE event of the patient corresponding to the MAP value and optionally the patient's personalized AHE/MAP threshold value(s). In one example, the apparatus is configured to determine four levels of severity: level 1 severity (L1) for a predicted MAP value of 50 mmHg or lower, level 2 severity (L2) for a predicted MAP value between 50 mmHg and 65 mmHg, level 3 severity (L3) for a predicted MAP value between 65 mmHg and 75 mmHg and level 4 severity (L4) for a predicted MAP value of above 75 mmHg.

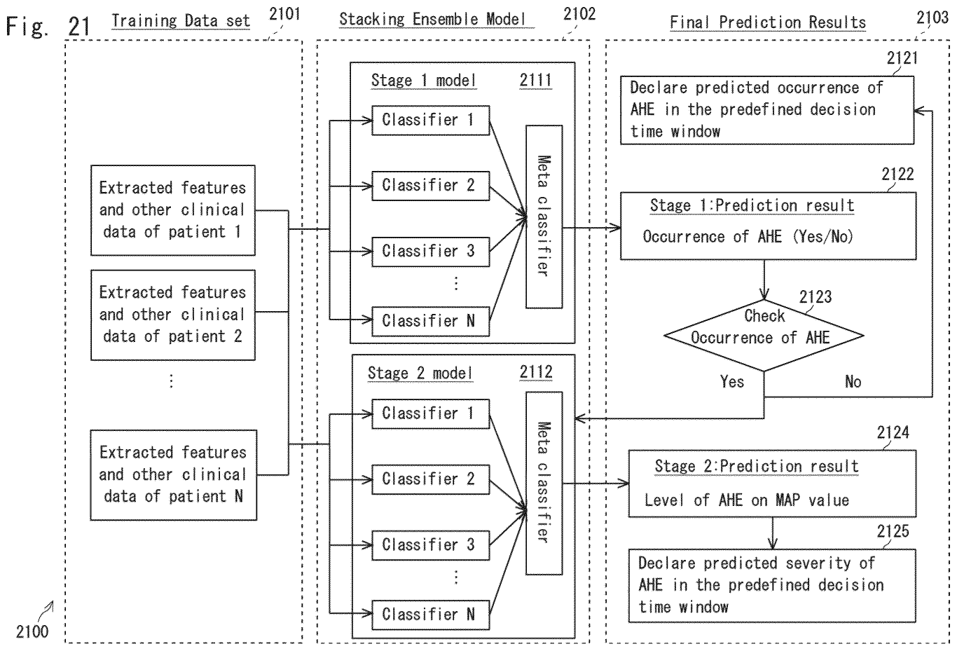

FIG. 21 shows a flow diagram illustrating a process for detecting and predicting an AHE event of a patient according an embodiment of the present disclosure. In this embodiment, a prediction of an occurrence of an AHE event of a patient is first determined in stage 1, or by stage 1 model 2111, and the followed by a prediction of a MAP value of the AHE event and a severity level is determined in stage 2, or by stage 2 model 2112. In this embodiment, both stage 1 model and stage 2 model adopt sequential multilevel classification where a plurality of classifiers is used to first generate a prediction in each of them and a meta classifier is then used to compare the generated predictions to determine an optimal prediction model (herein after referred to as “stage 1 model” and stage 2 model” respectively) for use in generating the final prediction results 2103.

Training data set 2101 comprising extracted features and other clinical data of a plurality of patients (patients 1, 2, . . . n) are input to each of multiple classifiers (classifiers 1, 2, . . . n) and then a meta classifier in the stage 1 model 2111 to determine a prediction result 2122 of an occurrence of an AHE event within a pre-defined decision time window. In this embodiment, a prediction result of “yes” or “no” is simply output.

In step 2123, it is checked if the prediction result of occurrence of an AHE event is “no”, step 2121 is carried out. In step 2121, the predicted occurrence of AHE in the predefined decision time window is declared. If the prediction result of occurrence of an AHE event is “yes”, stage 2 model 2112 is then applied to determine a prediction result 2124 of MAP value of the AHE event and a severity level of the AHE event based on the predicted MAP value. In step 2125, the predicted severity of the AHE event in the predefined decision time window is then declared.

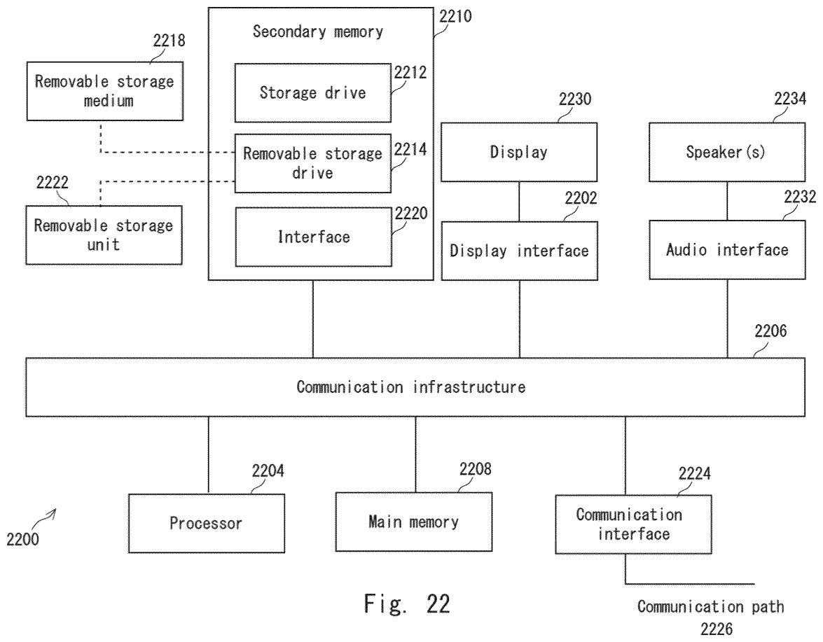

FIG. 22 depicts an exemplary computing device 2200, hereinafter interchangeably referred to as a computer system 2200, where one or more such computing devices 2200 may be used to execute the method of FIG. 1. The exemplary computing device 2200 can be used to implement the apparatus 200 shown in FIG. 2. The following description of the computing device 2200 is provided by way of example only and is not intended to be limiting.

As shown in FIG. 22, the example computing device 2200 includes a processor 2204 for executing software routines. Although a single processor is shown for the sake of clarity, the computing device 2200 may also include a multi-processor system. The processor 2204 is connected to a communication infrastructure 2206 for communication with other components of the computing device 2200. The communication infrastructure 2206 may include, for example, a communications bus, cross-bar, or network.

The computing device 2200 further includes a main memory 2208, such as a random access memory (RAM), and a secondary memory 2210. The secondary memory 2210 may include, for example, a storage drive 2212, which may be a hard disk drive, a solid state drive or a hybrid drive and/or a removable storage drive 2214, which may include a magnetic tape drive, an optical disk drive, a solid state storage drive (such as a USB flash drive, a flash memory device, a solid state drive or a memory card), or the like. The removable storage drive 2214 reads from and/or writes to a removable storage medium 2218 in a well-known manner. The removable storage medium 2218 may include magnetic tape, optical disk, non-volatile memory storage medium, or the like, which is read by and written to by removable storage drive 2214. As will be appreciated by persons skilled in the relevant art(s), the removable storage medium 2218 includes a computer readable storage medium having stored therein computer executable program code instructions and/or data.

In an alternative implementation, the secondary memory 2210 may additionally or alternatively include other similar means for allowing computer programs or other instructions to be loaded into the computing device 2200. Such means can include, for example, a removable storage unit 2222 and an interface 2220. Examples of a removable storage unit 2222 and interface 2220 include a program cartridge and cartridge interface (such as that found in video game console devices), a removable memory chip (such as an EPROM or PROM) and associated socket, a removable solid state storage drive (such as a USB flash drive, a flash memory device, a solid state drive or a memory card), and other removable storage units 2222 and interfaces 2220 which allow software and data to be transferred from the removable storage unit 2222 to the computer system 2200.

The computing device 2200 also includes at least one communication interface 2224. The communication interface 2224 allows software and data to be transferred between computing device 2200 and external devices via a communication path 2226. In various embodiments of the inventions, the communication interface 2224 permits data to be transferred between the computing device 2200 and a data communication network, such as a public data or private data communication network. The communication interface 2224 may be used to exchange data between different computing devices which such computing devices 2200 form part an interconnected computer network. Examples of a communication interface 2224 can include a modem, a network interface (such as an Ethernet card), a communication port (such as a serial, parallel, printer, GPIB, IEEE 1394, RJ45, USB), an antenna with associated circuitry and the like. The communication interface 2224 may be wired or may be wireless. Software and data transferred via the communication interface 2224 are in the form of signals which can be electronic, electromagnetic, optical or other signals capable of being received by communication interface 2224. These signals are provided to the communication interface via the communication path 2226.

As shown in FIG. 22, the computing device 2200 further includes a display interface 2202 which performs operations for rendering images to an associated display 2230 and an audio interface 2232 for performing operations for playing audio content via associated speaker(s) 2234.

As used herein, the term “computer program product” may refer, in part, to removable storage medium 2218, removable storage unit 2222, a hard disk installed in storage drive 2212, or a carrier wave carrying software over communication path 2226 (wireless link or cable) to communication interface 2224. Computer readable storage media refers to any non-transitory, non-volatile tangible storage medium that provides recorded instructions and/or data to the computing device 2200 for execution and/or processing. Examples of such storage media include magnetic tape, CD-ROM, DVD, Blu-ray Disc, a hard disk drive, a ROM or integrated circuit, a solid state storage drive (such as a USB flash drive, a flash memory device, a solid state drive or a memory card), a hybrid drive, a magneto-optical disk, or a computer readable card such as a PCMCIA card and the like, whether or not such devices are internal or external of the computing device 2200. Examples of transitory or non-tangible computer readable transmission media that may also participate in the provision of software, application programs, instructions and/or data to the computing device 2200 include radio or infra-red transmission channels as well as a network connection to another computer or networked device, and the Internet or Intranets including e-mail transmissions and information recorded on Websites and the like.

The computer programs (also called computer program code) are stored in main memory 2208 and/or secondary memory 2210. Computer programs can also be received via the communication interface 2224. Such computer programs, when executed, enable the computing device 2200 to perform one or more features of embodiments discussed herein. In various embodiments, the computer programs, when executed, enable the processor 1207 to perform features of the above-described embodiments. Accordingly, such computer programs represent controllers of the computer system 2200.

Software may be stored in a computer program product and loaded into the computing device 2200 using the removable storage drive 2214, the storage drive 2212, or the interface 2220. The computer program product may be a non-transitory computer readable medium. Alternatively, the computer program product may be downloaded to the computer system 2200 over the communications path 2226. The software, when executed by the processor 2204, causes the computing device 2200 to perform the necessary operations to execute the method as shown in FIG. 1.

It is to be understood that the embodiment of FIG. 22 is presented merely by way of example to explain the operation and structure of the apparatus 200. Therefore, in some embodiments one or more features of the computing device 2200 may be omitted. Also, in some embodiments, one or more features of the computing device 2200 may be combined together. Additionally, in some embodiments, one or more features of the computing device 2200 may be split into one or more component parts.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

Further, the whole or part of the embodiments disclosed above can be described as, but not limited to, the following supplementary notes.

### (Supplementary Note 1)

A method for adaptively detecting an Acute Hypotension Episode (AHE) event of a patient, comprising:
processing, by a processor, a signal relating to the patient to determine at least one mean arterial pressure (MAP) threshold value for the patient; and
detecting, by the processor, an AHE event of the patient based on the at least one MAP threshold value.

### (Supplementary Note 2)

The method according to Supplementary Note 1, wherein the step of processing, by the processor, the signal to determine the at least one mean arterial pressure (MAP) threshold value for the patient comprises:
determining at least one fluctuation of the signal from a baseline of the signal.

### (Supplementary Note 3)

The method according to Supplementary Note 1, wherein the step of detecting, by the processor, the AHE event of the patient comprises:
comparing one or more MAP values of the signal against the at least one MAP threshold value.

### (Supplementary Note 4)

The method according to Supplementary Note 3, wherein the step of detecting, by the processor, the AHE event of the patient comprises:
determining if a duration relating to the one or more MAP values is longer than a pre-configured threshold duration.

### (Supplementary Note 5)

The method according to Supplementary Note 1, wherein the step of detecting, by the processor, the AHE event of the patient comprises:
determining if the at least one MAP threshold value is lower than a first pre-configured MAP threshold value.

### (Supplementary Note 6)

The method according to Supplementary Note 1, wherein the step of detecting, by the processor, the AHE event of the patient comprises:
determining if a mean MAP value of a baseline of the signal is lower than a second pre-configured MAP threshold value.

### (Supplementary Note 7)

The method according to any one of the preceding Supplementary Notes, wherein the step of detecting, by the processor, the one or more AHE events of the patient comprises:

detecting an onset and a duration of the AHE event of the patient based on the at least one MAP threshold value and the signal.

### (Supplementary Note 8)

The method according to Supplementary Note 7, further comprising:
storing the onset and the duration of the AHE event of the patient, and one or more MAP values associated with the AHE event in a database.

### (Supplementary Note 9)

The method according to any one of the preceding Supplementary Notes, further comprising:
receiving a MAP waveform, wherein the signal comprises the MAP waveform.

### (Supplementary Note 10)

The method according to any one of the preceding Supplementary Notes, further comprising:
receiving at least one clinical data, the at least one clinical data comprising at least one of a blood composition, a urine composition, hemodynamic waveform data, demographic data, comorbidity data, a blood pressure, a central venous pressure, a frailty score and perioperative administration data, wherein the signal comprises the at least one clinal data.

### (Supplementary Note 11)

The method according to any one of the preceding Supplementary Notes, further comprising:
determining, by the processor, a prediction of a subsequent AHE event of the patient based on the at least one MAP threshold value and the signal.

### (Supplementary Note 12)

The method according to Supplementary Note 11, further comprising:
receiving an input relating to a time frame, wherein the prediction comprises a prediction of an occurrence of the subsequent AHE event of the patient within the time frame.

### (Supplementary Note 13)

The method according to Supplementary Note 11, wherein the step of determining, by the processor, the prediction of the subsequent AHE event of the patient comprises:
determining a MAP value of the subsequent AHE event, the MAP value relating to a severity level of the subsequent AHE event.

### (Supplementary Note 14)

The method according to any one of Supplementary Notes 11 to 13, further comprising:
extracting at least one feature in the signal, wherein the determination of the prediction of the subsequent AHE event is based on the at least one feature of the signal.

### (Supplementary Note 15)

The method according to Supplementary Note 14, wherein the step of determining, by the processor, the prediction of the subsequent AHE of the patient comprises:
determining two or more predictions based on the at least one feature extracted from a plurality of signals relating to a plurality of patients; and
comparing the two or more predictions to determine an optimal prediction of subsequent AHE events of the plurality of patients based on the at least one feature extracted from the plurality of signals, the optimal prediction being either one of or a combination of the two or more predictions, wherein the prediction of the subsequent AHE of the patient is based on the optimal prediction of the subsequent AHE events of the plurality of patients.

### (Supplementary Note 16)

An apparatus for adaptively detecting an Acute Hypotension Episode (AHE) event of a patient, comprising:
at least one processor; and
at least one memory including computer program code;
the at least one memory and the computer program code configured to, with at least one processor, cause the apparatus at least to perform the method according to any one of Supplementary Notes 1 to 15.

This application is based upon and claims the benefit of priority from Singapore patent application Ser. No. 10202108729V, filed on Aug. 10, 2021, the disclosure of which is incorporated herein in its entirety by reference.

[Reference Signs List]

| | |
|---|---|
| 100 | flow chart |
| 200 | block diagram |
| 201 | source |
| 202 | apparatus |
| 204 | personalized AHE definition module |
| 206 | hypotension episode prediction module |
| 208 | data storage |
| 210 | monitoring module |
| 211 | user interface |
| 300 | block diagram |
| 301 | signal source |
| 304 | personalized AHE definition module |
| 311 | data storage |
| 313 | patient monitoring unit |
| 314 | invasive blood pressure monitoring device |
| 315 | non-invasive or minimally invasive blood |
| | pressure monitoring device |
| 601 | MAP waveform |
| 602 | smoothened MAP waveform |
| 700 | processed MAP waveform |
| 800 | flow chart |
| 901 | processed MAP waveform |
| 1001 | processed MAP waveform |
| 1002 | subsample of the processed MAP waveform 1001 |
| 1003 | processed MAP waveform |
| 1101 | processed MAP waveform |
| 1102 | subsample of the processed MAP waveform 1101 |
| 1103 | processed MAP waveform |
| 1201 | processed MAP waveform |
| 1202 | subsample of the processed MAP waveform 1201 |
| 1203 | processed MAP waveform |
| 1401 | data storage |
| 1402 | personalized AHE definition module |
| 1403 | other clinical data |
| 1404 | hypotension prediction module |
| 1411 | user interface |
| 1600 | block diagram |
| 1601 | predictive modelling engine |
| 1602, 1603 | input |
| 1802, 1804 | signal |
| 1803, 1805 | table |
| 1908 | table |
| 2000 | apparatus |
| 2001 | input data to prediction modeling module |
| 2002 | predictive modelling module |
| 2003 | output of prediction model |
| 2101 | training data set |
| 2102 | stacking ensemble model |
| 2103 | final prediction results |
| 2111 | stage 1 model |
| 2112 | stage 2 model |
| 2200 | computing device |
| 2202 | display interface |
| 2204 | processor |
| 2206 | communication infrastructure |
| 2208 | main memory |
| 2210 | secondary memory |
| 2212 | storage drive |
| 2214 | removable storage drive |
| 2218 | removable storage medium |
| 2220 | interface |
| 2222 | removable storage unit |
| 2224 | communication interface |
| 2226 | communication path |
| 2230 | display |
| 2232 | audio interface |
| 2234 | speaker |

What is claimed is:

1. A method for adaptively detecting an Acute Hypotension Episode (AHE) event of a patient, comprising:
processing, by a processor, a signal relating to the patient to determine at least one mean arterial pressure (MAP) threshold value for the patient; and
detecting, by the processor, an AHE event of the patient based on the at least one MAP threshold value.

2. The method according to claim 1, wherein the step of processing, by the processor, the signal to determine the at least one MAP threshold value for the patient comprises:
determining at least one fluctuation of the signal from a baseline of the signal.

3. The method according to claim 1, wherein the detecting, by the processor, the AHE event of the patient comprises:
comparing one or more MAP values of the signal against the at least one MAP threshold value.

4. The method according to claim 3, wherein the detecting, by the processor, the AHE event of the patient comprises:
determining if a duration relating to the one or more MAP values is longer than a pre-configured threshold duration.

5. The method according to claim 1, wherein the detecting, by the processor, the AHE event of the patient comprises:
determining if the at least one MAP threshold value is lower than a first pre-configured MAP threshold value.

6. The method according to claim 1, wherein the detecting, by the processor, the AHE event of the patient comprises:
determining if a mean MAP value of a baseline of the signal is lower than a second pre-configured MAP threshold value.

7. The method according to claim 1, wherein the detecting, by the processor, the one or more AHE events of the patient comprises:
detecting an onset and a duration of the AHE event of the patient based on the at least one MAP threshold value and the signal.

8. The method according to claim 7, further comprising:
storing the onset and the duration of the AHE event of the patient, and one or more MAP values associated with the AHE event in a database.

9. The method according to claim 1, further comprising:
receiving a MAP waveform, wherein the signal comprises the MAP waveform.

10. The method according to claim 1, further comprising:
receiving at least one clinical data, the at least one clinical data comprising at least one of a blood composition, a urine composition, hemodynamic waveform data, demographic data, comorbidity data, a blood pressure, a central venous pressure, a frailty score and perioperative administration data, wherein the signal comprises the at least one clinal data.

11. The method according to claim 1, further comprising:
determining, by the processor, a prediction of a subsequent AHE event of the patient based on the at least one MAP threshold value and the signal.

12. The method according to claim 11, further comprising:
receiving an input relating to a time frame, wherein the prediction comprises a prediction of an occurrence of the subsequent AHE event of the patient within the time frame.

13. The method according to claim 11, wherein the determining, by the processor, the prediction of the subsequent AHE event of the patient comprises:
determining a MAP value of the subsequent AHE event, the MAP value relating to a severity level of the subsequent AHE event.

14. The method according to claim 11, further comprising:
extracting at least one feature in the signal, wherein the determination of the prediction of the subsequent AHE event is based on the at least one feature of the signal.

15. The method according to claim 14, wherein the determining, by the processor, the prediction of the subsequent AHE of the patient comprises:
determining two or more predictions based on the at least one feature extracted from a plurality of signals relating to a plurality of patients; and
comparing the two or more predictions to determine an optimal prediction of subsequent AHE events of the plurality of patients based on the at least one feature extracted from the plurality of signals, the optimal prediction being either one of or a combination of the two or more predictions, wherein the prediction of the subsequent AHE of the patient is based on the optimal prediction of the subsequent AHE events of the plurality of patients.

16. An apparatus for adaptively detecting an Acute Hypotension Episode (AHE) event of a patient, comprising:
at least one processor; and
at least one memory including computer program code;
the at least one memory and the computer program code configured to, with at least one processor, cause the apparatus at least to perform the method according to claim 1.
